(19) United States
(12) Patent Application Publication
(10) Pub. No.: US 2011/0112133 A1
(43) Pub. Date: May 12, 2011

(54) DIHYDRO PYRROLOQUINOLINE DERIVATIVES

(75) Inventors: Kasei Miura, Osaka (JP); Yuji Nishikimi, Osaka (JP)

(73) Assignee: Takeda Pharmaceutical Company Limited, Osaka (JP)

(21) Appl. No.: 12/916,721

(22) Filed: Nov. 1, 2010

(30) Foreign Application Priority Data
Nov. 6, 2009 (JP) 255574/2009
Apr. 15, 2010 (JP) 094144/2010

(51) Int. Cl. A61K 31/437; C07D 471/04; A61P 1/04; A61P 25/00

(52) U.S. Cl. 514/292; 546/84

(57) ABSTRACT

A compound represented by the formula (I)

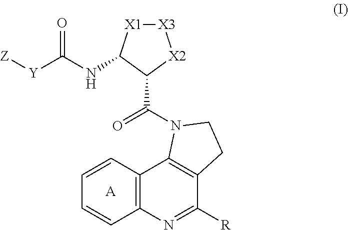

wherein A is a benzene ring optionally having substituent(s), R is a hydrogen atom, a hydrocarbon group optionally having substituent(s) or a heterocyclic group optionally having substituent(s), X1 and X2 are each a bond or a divalent C₁₋₅ chain hydrocarbon group optionally having substituent(s), X3 is a methylene group having substituent(s), Y is a bond or the like, and Z is a hydrocarbon group optionally having substituent(s) or the like, or a salt thereof. The compound of the present invention or a salt thereof is useful as an NK receptor antagonist.

### RELATED APPLICATIONS

This application claims priority to Japanese patent application No. 255574/2009, filed Nov. 6, 2009, and Japanese patent application Ser. No. 094144/2010, filed Apr. 15, 2010. The entire disclosures of each of the aforementioned patent applications are incorporated herein by this reference.

### TECHNICAL FIELD

The present invention relates to an optically active dihydro pyrroloquinoline derivative and use thereof.

### BACKGROUND OF THE INVENTION

Tachykinin is a generic term of a group of neuropeptides, and substance P (hereinafter SP), neurokinin A (hereinafter abbreviated as NKA) and neurokinin B (hereinafter abbreviated as NKB) in mammals are known. These peptides are known to be bound with receptors (neurokinin 1, neurokinin 2, neurokinin 3, hereinafter abbreviated as NK1, NK2, NK3, respectively) thereof present in vivo to exert various biological activities.

Particularly, the NK2 receptor antagonists are considered to be useful for the prophylaxis or treatment of neurokinin A dependent pathology, and they are considered to be useful for the prophylaxis or treatment of diseases such as pulmonary diseases (particularly, bronchospasm due to asthma, cough, chronic obstructive pulmonary disease and pulmonary anaphylaxis), gastrointestinal diseases (particularly, enterospasm, irritable bowel syndrome, inflammatory intestine diseases, non-ulcer dyspepsia, esophageal reflux and GI tract disorders), central nervous diseases (e.g., melancholia, anxiety), urinary diseases (e.g., dysuria), pain diseases (e.g., nervous pain, pain associated with inflammatory diseases such as rheumatism and the like) (Expert Opin. Ther. Targets, (2003) vol. 7(3), p. 343).

As such NK2 receptor antagonist, a hexahydro pyrroloquinoline derivative and a production method thereof are disclosed (WO2008/153027). In addition, as an NK2 receptor antagonist, a dihydro pyrroloquinoline derivative and a production method thereof are also disclosed (WO2005/105802).

However, a compound having more superior activity is desired.

### SUMMARY OF THE INVENTION

The present invention aims to provide a compound having a superior NK2 receptor antagonistic action.

The present inventors have conducted intensive studies and found that, among dihydro pyrroloquinoline derivatives, a compound having, as a side chain thereof, a cyclic hydrocarbon group having a substituent different from conventional ones shows a more superior NK2 receptor antagonistic action, which resulted in the completion of the present invention.

Accordingly, the present invention provides

[1] a compound represented by the formula (I)

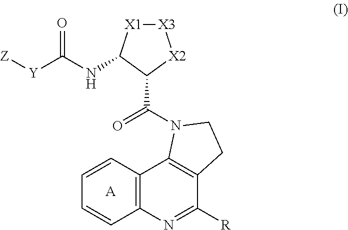

wherein
A is a benzene ring optionally having substituent(s),
R is a hydrogen atom, a hydrocarbon group optionally having substituent(s) or a heterocyclic group optionally having substituent(s),
X1 and X2 are each a bond or a divalent C₁₋₅ chain hydrocarbon group optionally having substituent(s),
X3 is a methylene group having substituent(s),
Y is a bond or an imino group (—NH—) optionally having a substituent, and
Z is a hydrocarbon group optionally having substituent(s) or a heterocyclic group optionally having substituent(s), or a salt thereof (hereinafter sometimes abbreviated as compound (I));
[2] the compound of the aforementioned [1], wherein X3 is a methylene group having substituent(s) selected from (1) a fluorine atom and (2) a C₁₋₃ alkyl group substituted by fluorine atom(s), or a salt thereof;
[3] the compound of the aforementioned [1], wherein X1 is ethylene (—CH₂CH₂—) and X2 is methylene (—CH₂—), or a salt thereof;
[4] the compound of the aforementioned [2], wherein X3 is a methylene group having fluorine atom(s), or a salt thereof;
[5] the compound of the aforementioned [1], wherein R is an aromatic hydrocarbon group optionally having substituent(s) or an aromatic heterocyclic group optionally having substituent(s), or a salt thereof;
[6] the compound of the aforementioned [1], wherein A is a benzene ring optionally substituted by fluorine atom(s), or a salt thereof;
[7] the compound of the aforementioned [1], wherein Y is a bond or an imino group (—NH—), or a salt thereof;
[8] the compound of the aforementioned [1], wherein Z is an aromatic hydrocarbon group optionally having substituent(s) or an aromatic heterocyclic group optionally having substituent(s), or a salt thereof;
[9] N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide or a salt thereof;
[10] N-{(1R,2S)-4,4-difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide or a salt thereof;
[11] N-{(1R,2S)-4,4-difluoro-2-[(8-fluoro-4-phenyl-2,3-dihydro-1H-pyrrolo[3,2-c]quinolin-1-yl)carbonyl]cyclohexyl}-4-(3-methyl-1H-pyrazol-1-yl)benzamide or a salt thereof;
[12] a prodrug of the compound of the aforementioned [1] or a salt thereof;
[13] a neurokinin (NK) receptor antagonist comprising the compound of the aforementioned [1] or a salt thereof, or a prodrug thereof;
[14] a neurokinin 2 (NK2) receptor antagonist comprising the compound of the aforementioned [1] or a salt thereof, or a prodrug thereof;
[15] a medicament comprising the compound of the aforementioned [1] or a salt thereof, or a prodrug thereof;
[16] the medicament of the aforementioned [15], which is an agent for the prophylaxis or treatment of a gastrointestinal disease or a central nervous system disease;
[17] the medicament of the aforementioned [16], wherein the gastrointestinal disease is a functional gastrointestinal disease;
[18] the medicament of the aforementioned [17], wherein the functional gastrointestinal disease is irritable bowel syndrome or functional dyspepsia;
[19] a method for preventing or treating a gastrointestinal disease or a central nervous system disease, comprising administering an effective amount of the compound of the aforementioned [1] or a salt thereof, or a prodrug thereof to a mammal;
[20] the method of the aforementioned [19], wherein the gastrointestinal disease is a functional gastrointestinal disease;
[21] the method of the aforementioned [20], wherein the functional gastrointestinal disease is irritable bowel syndrome or functional dyspepsia;
[22] use of the compound of the aforementioned [1] or a salt thereof, or a prodrug thereof for the production of an agent for the prophylaxis or treatment of a gastrointestinal disease or a central nervous system disease;
[23] the use of the aforementioned [22], wherein the gastrointestinal disease is a functional gastrointestinal disease;
[24] the use of the aforementioned [23], wherein the functional gastrointestinal disease is irritable bowel syndrome or functional dyspepsia;
[25] a method for antagonizing an NK2 receptor, comprising administering an effective amount of the compound of the aforementioned [1] or a salt thereof, or a prodrug thereof to a mammal;
[26] use of the compound of the aforementioned [1] or a salt thereof, or a prodrug thereof for the production of an NK2 receptor antagonist; and
[27] the compound of the aforementioned [1] or a salt thereof, or a prodrug thereof for use in the prophylaxis or treatment of a gastrointestinal disease or a central nervous system disease.

The present invention provides a dihydro pyrroloquinoline derivative, which is compound (I) useful as an NK receptor antagonist, particularly an NK2 receptor antagonist.

### DETAILED DESCRIPTION OF THE INVENTION

The present invention is explained in detail in the following.

The “benzene ring optionally having substituent(s)” for A is a substituted or unsubstituted benzene ring. Examples of the substituents include

(1) a halogen atom;
(2) a cyano group;
(3) a hydroxyl group;
(4) a carbamoyl group;
(5) a C₁₋₃ alkyl group optionally substituted by one or more (preferably 1 to 3) substituents selected from the group consisting of a halogen atom, a hydroxyl group, a cyano group and a carbamoyl group; and
(6) a C₁₋₃ alkoxy group optionally substituted by one or more (preferably 1 to 3) substituents selected from the group consisting of a halogen atom, a hydroxyl group, a cyano group and a carbamoyl group.

The “halogen atom” is fluorine, chlorine, bromine, iodine or the like.

The “C₁₋₃ alkyl group” is a straight chain or branched alkyl group having a carbon number of 1 to 3, and a methyl group, an ethyl group, a propyl group and an isopropyl group can be mentioned.

The “C₁₋₃ alkoxy group” is a straight chain or branched alkoxy group having a carbon number of 1 to 3, and a methoxy group, an ethoxy group, a propoxy group and an isopropoxy group can be mentioned.

Examples of the “C₁₋₃ alkyl group optionally substituted by one or more (preferably 1 to 3) substituents selected from the group consisting of a halogen atom, a hydroxyl group, a cyano group and a carbamoyl group” include a methyl group, an ethyl group, a monofluoromethyl group, a difluoromethyl group, a trifluoromethyl group, a cyanomethyl group, a carbamoylmethyl group, a carbamoyldifluoromethyl group, a 2-cyanoethyl group, a 2-carbamoylethyl group, a 2,2,2-trifluoroethyl group, a hydroxymethyl group, a 2-hydroxyethyl group, a difluorohydroxymethyl group, an 1-hydroxyethyl group, a 3-hydroxypropyl group and the like.

Examples of the “C₁₋₃ alkoxy group optionally substituted by one or more (preferably 1 to 3) substituents selected from the group consisting of a halogen atom, a hydroxyl group, a cyano group and a carbamoyl group” include a methoxy group, an ethoxy group, a monofluoromethoxy group, a difluoromethoxy group, a trifluoromethoxy group, a cyanomethoxy group, a carbamoylmethoxy group, a cyanodifluoromethoxy group, a carbamoyldifluoromethoxy group, a 2-cyanoethoxy group, a 2-carbamoylethoxy group, a 2,2,2-trifluoroethoxy group, a 2-hydroxyethoxy group, a 3-hydroxypropoxy group and the like.

As the substituent of the benzene ring of the “benzene ring optionally having substituent(s)”,

(1) a halogen atom;
(2) a hydroxyl group;
(3) a C₁₋₃ alkyl group optionally substituted by one or more (preferably 1 to 3) substituents selected from the group consisting of a halogen atom, a hydroxyl group, a cyano group and a carbamoyl group; or
(4) a C₁₋₃ alkoxy group optionally substituted by one or more (preferably 1 to 3) substituents selected from the group consisting of a halogen atom, a hydroxyl group, a cyano group and a carbamoyl group is preferable.
More preferred is a fluorine atom, a chlorine atom, a bromine atom, a hydroxyl group, a methyl group, an ethyl group, a monofluoromethyl group, a difluoromethyl group, a trifluoromethyl group, a 2,2,2-trifluoroethyl group, a hydroxymethyl group, a methoxy group, an ethoxy group, a monofluoromethoxy group, a difluoromethoxy group, a trifluoromethoxy group, a 2,2,2-trifluoroethoxy group or a 2-hydroxyethoxy group, and further preferred is a fluorine atom, a chlorine atom, a bromine atom, a hydroxyl group, a methyl group or a methoxy group.

The position of substitution of the substituent may be any substitutable position, and the 8-position in the following formula (I) (the 8-position of 2,3-dihydro-1H-pyrrolo[3,2-c]quinoline ring) is more preferable.

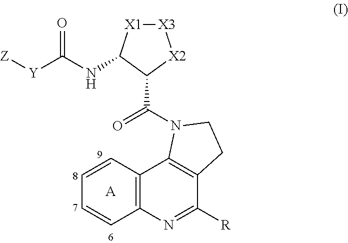

The number of the substituents can be 0 to 4, not more than 2 is preferable, and 0 or 1 is more preferable.

The “heterocyclic group optionally having substituent(s)” for R or Z is a substituted or unsubstituted heterocyclic group. The “heterocyclic group” is a 3- to 14-membered (monocyclic, bicyclic or tricyclic) heterocyclic group containing, besides carbon atom, 1 to 5 hetero atoms of 1 to 3 kinds selected from a nitrogen atom, a sulfur atom and an oxygen atom, and a nonaromatic heterocyclic group or an aromatic heterocyclic group.

The “nonaromatic heterocyclic group” is a monocyclic, bicyclic or tricyclic heterocyclic group having no aromaticity, and an oxazolidinyl group (a 2-oxazolidinyl group, a 4-oxazolidinyl group, a 5-oxazolidinyl group etc.), an imidazolidinyl group (a 1-imidazolidinyl group, a 2-imidazolidinyl group, a 4-imidazolidinyl group etc.), an imidazolinyl group (an 1-imidazolinyl group, a 2-imidazolinyl group, a 4-imidazolinyl group etc.), an aziridinyl group (a 1-aziridinyl group, a 2-aziridinyl group etc.), an azetidinyl group (a 1-azetidinyl group, a 2-azetidinyl group etc.), a pyrrolidinyl group (a 1-pyrrolidinyl group, a 2-pyrrolidinyl group, a 3-pyrrolidinyl group etc.), a piperidinyl group (a 1-piperidinyl group, a 2-piperidinyl group, a 3-piperidinyl group etc.), an azepanyl group (a 1-azepanyl group, a 2-azepanyl group, a 3-azepanyl group, a 4-azepanyl group etc.), an azocanyl group (a 1-azocanyl group, a 2-azocanyl group, a 3-azocanyl group, a 4-azocanyl group etc.), a piperazinyl group (a 1,4-piperazin-1-yl group, a 1,4-piperazin-2-yl group etc.), a diazepanyl group (a 1,4-diazepan-1-yl group, a 1,4-diazepan-2-yl group, a 1,4-diazepan-5-yl group, a 1,4-diazepan-6-yl group etc.), a diazocanyl group (a 1,4-diazocan-1-yl group, a 1,4-diazocan-2-yl group, a 1,4-diazocan-5-yl group, a 1,4-diazocan-6-yl group, a 1,5-diazocan-1-yl group, a 1,5-diazocan-2-yl group, a 1,5-diazocan-3-yl group etc.), a 4-morpholinyl group, a 4-thiomorpholinyl group, an indolinyl group, a dihydroquinolyl group and the like can be mentioned.

The “aromatic heterocyclic group” is a monocyclic, bicyclic or tricyclic heterocyclic group having aromaticity, and a pyrrolyl group (a 1-pyrrolyl group, a 2-pyrrolyl group, a 3-pyrrolyl group etc.), a furyl group (a 2-furyl group, a 3-furyl group etc.), a thienyl group (a 2-thienyl group, a 3-thienyl group etc.), a pyrazolyl group (a 1-pyrazolyl group, a 3-pyrazolyl group, a 4-pyrazolyl group etc.), an imidazolyl group (a 1-imidazolyl group, a 2-imidazolyl group, a 4-imidazolyl group etc.), an isoxazolyl group (a 3-isoxazolyl group, a 4-isoxazolyl group, a 5-isoxazolyl group etc.), an oxazolyl group (a 2-oxazolyl group, a 4-oxazolyl group, a 5-oxazolyl group etc.), an isothiazolyl group (a 3-isothiazolyl group, a 4-isothiazolyl group, a 5-isothiazolyl group etc.), a thiazolyl group (a 2-thiazolyl group, a 4-thiazolyl group, a 5-thiazolyl group etc.), a triazolyl group (a 1,2,3-triazol-4-yl group, a 1,2,4-triazol-3-yl group etc.), an oxadiazolyl group (a 1,2,4-oxadiazol-3-yl group, a 1,2,4-oxadiazol-5-yl group etc.), a thiadiazolyl group (a 1,2,4-thiadiazol-3-yl group, a 1,2,4-thiadiazol-5-yl group etc.), a tetrazolyl group, a pyridyl group (a 2-pyridyl group, a 3-pyridyl group, a 4-pyridyl group etc.), a pyridazinyl group (a 3-pyridazinyl group, a 4-pyridazinyl group etc.), a pyrimidinyl group (a 2-pyrimidinyl group, a 4-pyrimidinyl group etc.), a pyrazinyl group, an isoindolyl group (a 1-isoindolyl group, a 2-isoindolyl group, a 3-isoindolyl group, a 4-isoindolyl group, a 5-isoindolyl group, a 6-isoindolyl group, a 7-isoindolyl group etc.), an indolyl group (a 1-indolyl group, a 2-indolyl group, a 3-indolyl group, a 4-indolyl group, a 5-indolyl group, a 6-indolyl group, a 7-indolyl group etc.), a benzo[b]furanyl group (a 2-benzo[b]furanyl group, a 3-benzo[b]furanyl group, a 4-benzo[b]furanyl group, a 5-benzo[b]furanyl group, a 6-benzo[b]furanyl group, a 7-benzo[b]furanyl group etc.), a benzo[c]furanyl group (a 1-benzo[c]furanyl group, a 4-benzo[c]furanyl group, a 5-benzo[c]furanyl group etc.), a benzo[b]thienyl group (a 2-benzo[b]thienyl group, a 3-benzo[b]thienyl group, a 4-benzo[b]thienyl group, a 5-benzo[b]thienyl group, a 6-benzo[b]thienyl group, a 7-benzo[b]thienyl group etc.), a benzo[c]thienyl group (a 1-benzo[c]thienyl group, a 4-benzo[c]thienyl group, a 5-benzo[c]thienyl group etc.), an indazolyl group (a 1-indazolyl group, a 2-indazolyl group, a 3-indazolyl group, a 4-indazolyl group, a 5-indazolyl group, a 6-indazolyl group, a 7-indazolyl group etc.), a benzimidazolyl group (a 1-benzimidazolyl group, a 2-benzimidazolyl group, a 4-benzimidazolyl group, a 5-benzimidazolyl group etc.), a 1,2-benzisoxazolyl group (a 1,2-benzisoxazol-3-yl group, a 1,2-benzisoxazol-4-yl group, a 1,2-benzisoxazol-5-yl group, a 1,2-benzisoxazol-6-yl group, a 1,2-benzisoxazol-7-yl group etc.), a benzoxazolyl group (a 2-benzoxazolyl group, a 4-benzoxazolyl group, a 5-benzoxazolyl group, a 6-benzoxazolyl group, a 7-benzoxazolyl group etc.), a 1,2-benzisothiazolyl group (a 1,2-benzisothiazol-3-yl group, a 1,2-benzisothiazol-4-yl group, a 1,2-benzisothiazol-5-yl group, a 1,2-benzisothiazol-6-yl group, a 1,2-benzisothiazol-7-yl group etc.), a benzothiazolyl group (a 2-benzothiazolyl group, a 4-benzothiazolyl group, a 5-benzothiazolyl group, a 6-benzothiazolyl group, a 7-benzothiazolyl group etc.), an isoquinolyl group (a 1-isoquinolyl group, a 3-isoquinolyl group, a 4-isoquinolyl group, a 5-isoquinolyl group etc.), a quinolyl group (a 2-quinolyl group, a 3-quinolyl group, a 4-quinolyl group, a 5-quinolyl group, a 8-quinolyl group etc.), a cinnolinyl group (a 3-cinnolinyl group, a 4-cinnolinyl group, a 5-cinnolinyl group, a 6-cinnolinyl group, a 7-cinnolinyl group, a 8-cinnolinyl group etc.), a phthalazinyl group (a 1-phthalazinyl group, a 4-phthalazinyl group, a 5-phthalazinyl group, a 6-phthalazinyl group, a 7-phthalazinyl group, a 8-phthalazinyl group etc.), a quinazolinyl group (a 2-quinazolinyl group, a 4-quinazolinyl group, a 5-quinazolinyl group, a 6-quinazolinyl group, a 7-quinazolinyl group, a 8-quinazolinyl group etc.), a quinoxalinyl group (a 2-quinoxalinyl group, a 3-quinoxalinyl group, a 5-quinoxalinyl group, a 6-quinoxalinyl group, a 7-quinoxalinyl group, a 8-quinoxalinyl group etc.), a pyrazolo[1,5-a]pyridyl group (a pyrazolo[1,5-a]pyridin-2-yl group, a pyrazolo[1,5-a]pyridin-3-yl group, a pyrazolo[1,5-a]pyridin-4-yl group, a pyrazolo[1,5-a]pyridin-5-yl group, a pyrazolo[1,5-a]pyridin-6-yl group, a pyrazolo[1,5-a]pyridin-7-yl group etc.), an imidazo[1,2-a]pyridyl group (an imidazo[1,2-a]pyridin-2-yl group, an imidazo[1,2-a]pyridin-3-yl group, an imidazo[1,2-a]pyridin-5-yl group, an imidazo[1,2-a]pyridin-6-yl group, an imidazo[1,2-a]pyridin-7-yl group, an imidazo[1,2-a]pyridin-8-yl group etc.) and the like can be mentioned.

Preferable examples of the “nonaromatic heterocyclic group” include a 2-oxazolidinyl group, a 4-oxazolidinyl group, a 5-oxazolidinyl group, a 1-imidazolidinyl group, a 2-imidazolidinyl group, a 4-imidazolidinyl group, a 1-imidazolinyl group, a 2-imidazolinyl group, a 4-imidazolinyl group, a 1-pyrrolidinyl group, a 1-piperidinyl group, a 1-azepanyl group, a 1,4-piperazin-1-yl group, a 4-morpholinyl group, a 4-thiomorpholinyl group, an indolinyl group, a dihydroquinolyl group and the like, more preferably, a 1-pyrrolidinyl group, a 1-piperidinyl group, a 1,4-piperazin-1-yl group and a 4-morpholinyl group.

Preferable examples of the “aromatic heterocyclic group” include a 1-pyrazolyl group, a 3-pyrazolyl group, a 1-imidazolyl group, a 2-imidazolyl group, a 4-imidazolyl group, a 2-thienyl group, a 3-thienyl group, a 1-pyrrolyl group, a 4-thiazolyl group, a 1,2,4-triazol-1-yl group, a 2-oxazolyl group, a 5-oxazolyl group, a 4-isoxazolyl group and the like, more preferably, a 3-pyrazolyl group, a 2-imidazolyl group, a 2-thienyl group, a 3-thienyl group, a 4-thiazolyl group, a 1,2,4-triazol-1-yl group, a 2-oxazolyl group and a 4-isoxazolyl group.

As the heterocyclic group, an aromatic heterocyclic group is preferable.

Examples of the substituent of the heterocyclic group of the “heterocyclic group optionally having substituent(s)” include

(1) a halogen atom;
(2) a cyano group;
(3) a hydroxyl group;
(4) a carbamoyl group;
(5) a C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkylcarbonyloxy group;
(6) a C₁₋₃ alkoxy group optionally substituted by one or more (preferably 1 to 3) substituents selected from the group consisting of a halogen atom, a hydroxyl group, a cyano group, a carbamoyl group and a C₁₋₆ alkyl-carbonyloxy group;
(7) a C₃₋₁₄ cyclic hydrocarbon group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkylcarbonyloxy group; and
(8) a heterocyclic group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group.

The “halogen atom”, “C₁₋₃ alkyl group”, “C₁₋₃ alkoxy group” and “heterocyclic group” mean the same as those exemplified for the aforementioned “benzene ring optionally having substituent(s)”.

The “C₁₋₆ alkyl-carbonyloxy group” is a carbonyloxy group substituted by a straight chain or branched alkyl group having a carbon number of 1 to 6, and an acetyloxy group, a propanoyloxy group, a butanoyloxy group, a pivaloyloxy group, a pentanoyloxy group, a hexanoyloxy group, a heptanoyloxy group and the like can be mentioned.

The “C₃₋₁₄ cyclic hydrocarbon group” is a monocyclic or polycyclic, and aromatic or nonaromatic hydrocarbon group having a carbon number of 3 to 14, and a phenyl group, a 1-naphthyl group, a 2-naphthyl group, a 2-anthryl group, a tetrahydronaphthyl group, a cyclopropyl group, a cyclobutyl group, a cyclopentyl group, a cyclohexyl group, a 1-adamantyl group, a 1-indanyl group, a 2-indanyl group, a 4-indanyl group, a 1-bicyclo[2.2.1]heptanyl group, a 2-bicyclo[2.2.1]heptanyl group, a 7-bicyclo[2.2.1]heptanyl group and the like can be mentioned. Preferred is an aromatic hydrocarbon group, more preferred is a phenyl group, a 1-naphthyl group or a 2-naphthyl group, and further preferred is a phenyl group.

Examples of the “C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkyl-carbonyloxy group” include a methyl group, an ethyl group, a cyanomethyl group, a carbamoylmethyl group, a carbamoyldifluoromethyl group, a monofluoromethyl group, a difluoromethyl group, a trifluoromethyl group, a 2,2,2-trifluoroethyl group, a hydroxymethyl group, a difluorohydroxymethyl group, a 2-hydroxyethyl group, a 1-hydroxyethyl group, a 3-hydroxypropyl group, an acetoxymethyl group, a pivaloyloxymethyl group and the like.

Examples of the “C₁₋₃ alkoxy group optionally substituted by one or more (preferably 1 to 3) substituents selected from the group consisting of a halogen atom, a hydroxyl group, a cyano group, a carbamoyl group and a C₁₋₆ alkyl-carbonyloxy group” include a methoxy group, an ethoxy group, a cyanomethoxy group, a carbamoylmethoxy group, a cyanodifluoromethoxy group, a carbamoyldifluoromethoxy group, an acetoxyethoxy group, a monofluoromethoxy group, a difluoromethoxy group, a trifluoromethoxy group, a 2,2,2-trifluoroethoxy group, a 2-hydroxyethoxy group, a 3-hydroxypropoxy group and the like.

Examples of the “C₃₋₁₄ cyclic hydrocarbon group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group” include a phenyl group, a 4-chlorophenyl group, a 4-fluorophenyl group, a 4-hydroxyphenyl group, a 2,3-dimethoxyphenyl group, a cyclopropyl group, a 1-adamantyl group and the like. The position of substitution of the substituent may be any substitutable position, and the number of the substituents is preferably 0 to 2, more preferably 0 or 1.

Examples of the “heterocyclic group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group” include a 1-pyrazolyl group, a 3-pyrazolyl group, a 3-methyl-1-pyrazolyl group, a 1-imidazolyl group, a 2-imidazolyl group, a 4-imidazolyl group, a 4-methyl-1-imidazolyl group, a 2-thienyl group, a 5-chloro-2-thienyl group, a 3-thienyl group, a 5-chloro-3-thienyl group, a 1-pyrrolyl group, a 1-piperidinyl group, a 2-methyl-4-thiazolyl group, a 1,2,4-triazol-1-yl group, a 2-oxazolyl group, a 5-oxazolyl group, a 2-methyl-5-oxazolyl group, a 3-methyl-4-isoxazolyl group and the like. The position of substitution of the substituent may be any substitutable position, and the number of the substituents is preferably 0 to 2, more preferably 0 or 1.

As the substituent of the heterocyclic group of the “heterocyclic group optionally having substituent(s)”,

(1) a halogen atom;
(2) a hydroxyl group;
(3) a C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkylcarbonyloxy group;
(4) a C₁₋₃ alkoxy group optionally substituted by one or more (preferably 1 to 3) substituents selected from the group consisting of a halogen atom, a hydroxyl group, a cyano group, a carbamoyl group and a C₁₋₆ alkyl-carbonyloxy group;
(5) a C₃₋₁₄ cyclic hydrocarbon group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkylcarbonyloxy group; or
(6) a heterocyclic group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group is preferable.
More preferred is fluorine, chlorine, bromine, a methyl group, an ethyl group, a trifluoromethyl group, a difluoromethyl group, a hydroxymethyl group, a 2-hydroxyethyl group, an acetoxymethyl group, a pivaloyloxymethyl group, a methoxy group, an ethoxy group, a trifluoromethoxy group, a difluoromethoxy group, a propoxy group, an isopropoxy group, a phenyl group, a 1-pyrazolyl group, a 3-pyrazolyl group, a 3-methyl-1-pyrazolyl group, a 1-imidazolyl group, a 2-imidazolyl group, a 4-imidazolyl group, a 4-methyl-1-imidazolyl group, a 2-thienyl group, a 3-thienyl group, a 1-pyrrolyl group, a 2-methyl-4-thiazolyl group, a 1,2,4-triazol-1-yl group, a 2-oxazolyl group, a 5-oxazolyl group, a 2-methyl-5-oxazolyl group or a 3-methyl-4-isoxazolyl group, and
further preferred is fluorine, a methyl group, a methoxy group, a trifluoromethoxy group, a difluoromethoxy group, a pivaloyloxymethyl group, a 1-pyrazolyl group, a 3-methyl-1-pyrazolyl group, a 2-methyl-4-thiazolyl group, a 1,2,4-triazol-1-yl group, a 2-oxazolyl group, a 5-oxazolyl group, a 2-methyl-5-oxazolyl group or a 3-methyl-4-isoxazolyl group. The position of substitution of the substituent may be any substitutable position, and the number of the substituents is preferably 0 to 2, more preferably 0 or 1.

The “hydrocarbon group optionally having substituent(s)” for R or Z is a substituted or unsubstituted hydrocarbon group. The “hydrocarbon group” is a C₁₋₆ alkyl group, a C₂₋₆ alkenyl group, a C₂₋₆ alkynyl group, a C₃₋₆ cycloalkyl group and an aromatic hydrocarbon group.

The “C₁₋₆ alkyl group” is a straight chain or branched alkyl group having a carbon number of 1 to 6, and a methyl group, an ethyl group, a propyl group, an isopropyl group, a butyl group, an isobutyl group, a sec-butyl group, a tert-butyl group, a pentyl group, an isopentyl group, a neopentyl group, a hexyl group and the like can be mentioned.

The “C₂₋₆ alkenyl group” is a straight chain or branched alkenyl group having carbon numbers of 2 to 6, and an ethenyl group, a 1-propenyl group, a 2-propenyl group and the like can be mentioned.

The “C₂₋₆ alkynyl group” is a straight chain or branched alkynyl group having carbon numbers of 2 to 6, and an ethynyl group, a 1-propynyl group, a 2-propynyl group and the like can be mentioned.

The “C₃₋₈ cycloalkyl group” is a cycloalkyl group having carbon numbers of 3 to 8, and a cyclopropyl group, a cyclobutyl group, a cyclopentyl group, a cyclohexyl group, a 1-bicyclo[2.2.1]heptanyl group, a 2-bicyclo[2.2.1]heptanyl group, a 7-bicyclo[2.2.1]heptanyl group and the like can be mentioned.

The “aromatic hydrocarbon group” is a monocyclic, bicyclic or tricyclic hydrocarbon group having carbon numbers of 6 to 14 having aromaticity, and a C₆₋₁₄ aryl group such as a phenyl group, a 1-naphthyl group, a 2-naphthyl group, a 2-anthryl group and the like can be mentioned.

Examples of the substituent of the hydrocarbon group in the “hydrocarbon group optionally having substituent(s)” include

(1) a halogen atom;
(2) a cyano group;
(3) a hydroxyl group;
(4) a carbamoyl group;
(5) a C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkylcarbonyloxy group;
(6) a C₁₋₃ alkoxy group optionally substituted by one or more (preferably 1 to 3) substituents selected from the group consisting of a halogen atom, a hydroxyl group, a cyano group, a carbamoyl group and a C₁₋₆ alkyl-carbonyloxy group;
(7) a C₃₋₁₄ cyclic hydrocarbon group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkylcarbonyloxy group; and
(8) a heterocyclic group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group.

The “halogen atom”, “C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkyl-carbonyloxy group”, “C₁₋₃ alkoxy group optionally substituted by one or more (preferably 1 to 3) substituents selected from the group consisting of a halogen atom, a hydroxyl group, a cyano group, a carbamoyl group and a C₁₋₆ alkyl-carbonyloxy group”, “C₁₋₆ alkyl-carbonyloxy group”, “C₃₋₁₄ cyclic hydrocarbon group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkylcarbonyloxy group” and “heterocyclic group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group” mean the same as those exemplified for the aforementioned “benzene ring optionally having substituent(s)” or those exemplified for the aforementioned “heterocyclic group optionally having substituent(s)”.

As the substituent of the hydrocarbon group of the “hydrocarbon group optionally having substituent(s)”,

(1) a halogen atom;
(2) a C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkylcarbonyloxy group;
(3) a C₁₋₃ alkoxy group optionally substituted by one or more (preferably 1 to 3) substituents selected from the group consisting of a halogen atom, a hydroxyl group, a cyano group, a carbamoyl group and a C₁₋₆ alkyl-carbonyloxy group;
(4) a C₃₋₁₄ cyclic hydrocarbon group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkylcarbonyloxy group; or
(5) a heterocyclic group optionally substituted by one or more substituents selected from the group consisting of a halogen atom, a hydroxyl group, a cyano group, a carbamoyl group, a C₁₋₃ alkyl group, a C₁₋₃ alkoxy group and a C₁₋₆ alkyl-carbonyloxy group is preferable.
More preferred is fluorine, chlorine, bromine, a methyl group, an ethyl group, a trifluoromethyl group, a difluoromethyl group, a hydroxymethyl group, a 2-hydroxyethyl group, an acetoxymethyl group, a pivaloyloxymethyl group, a methoxy group, an ethoxy group, a trifluoromethoxy group, a difluoromethoxy group, a propoxy group, an isopropoxy group, a phenyl group, a 1-pyrazolyl group, a 3-pyrazolyl group, a 3-methyl-1-pyrazolyl group, a 1-imidazolyl group, a 2-imidazolyl group, a 4-imidazolyl group, a 4-methyl-1-imidazolyl group, a 2-thienyl group, a 3-thienyl group, a 1-pyrrolyl group, a 2-methyl-4-thiazolyl group, a 1,2,4-triazol-1-yl group, a 2-oxazolyl group, a 5-oxazolyl group, a 2-methyl-5-oxazolyl group or a 3-methyl-4-isoxazolyl group, and
further preferred is fluorine, a methyl group, a methoxy group, a trifluoromethoxy group, a difluoromethoxy group, a pivaloyloxymethyl group, a 1-pyrazolyl group, a 3-methyl-1-pyrazolyl group, a 2-methyl-4-thiazolyl group, a 1,2,4-triazol-1-yl group, a 2-oxazolyl group, a 5-oxazolyl group, a 2-methyl-5-oxazolyl group or a 3-methyl-4-isoxazolyl group. The position of substitution of the substituent may be any substitutable position, and the number of the substituents is preferably 0 to 2, more preferably 0 or 1.

The “divalent C₁₋₅ chain hydrocarbon group optionally having substituent(s)” for X1 or X2 is a substituted or unsubstituted divalent C₁₋₅ chain hydrocarbon group. The “divalent C₁₋₅ chain hydrocarbon group” is a divalent chain hydrocarbon group having a carbon number of 1 to 5, and a methylene group (—CH₂—), an ethylene group (—(CH₂)₂—), a propylene group (—(CH₂)₃—), a butylene group (—(CH₂)₄—), a pentylene group (—(CH₂)₅—) and the like can be mentioned.

Examples of the substituent of the C₁₋₅ chain hydrocarbon group of the “divalent C₁₋₅ chain hydrocarbon group optionally having substituent(s)” include

(1) a halogen atom;
(2) a cyano group;
(3) a hydroxyl group;
(4) a carbamoyl group;
(5) a C₁₋₃ alkyl group optionally substituted by one or more (preferably 1 to 3) substituents selected from the group consisting of a halogen atom, a hydroxyl group, a cyano group and a carbamoyl group; and
(6) a C₁₋₃ alkoxy group optionally substituted by one or more (preferably 1 to 3) substituents selected from the group consisting of a halogen atom, a hydroxyl group, a cyano group and a carbamoyl group.

The “halogen atom”, “C₁₋₃ alkyl group optionally substituted by one or more (preferably 1 to 3) substituents selected from the group consisting of a halogen atom, a hydroxyl group, a cyano group and a carbamoyl group” and “C₁₋₃ alkoxy group optionally substituted by one or more (preferably 1 to 3) substituents selected from the group consisting of a halogen atom, a hydroxyl group, a cyano group and a carbamoyl group” mean the same as those exemplified for the aforementioned “benzene ring optionally having substituent(s)”.

As the substituent of the C₁₋₅ chain hydrocarbon group of the “divalent C₁₋₅ chain hydrocarbon group optionally having substituent(s)”,

(1) a halogen atom;
(2) a hydroxyl group;
(3) a C₁₋₃ alkyl group optionally substituted by one or more (preferably 1 to 3) substituents selected from the group consisting of a halogen atom, a hydroxyl group, a cyano group and a carbamoyl group; or
(4) a C₁₋₃ alkoxy group optionally substituted by one or more (preferably 1 to 3) substituents selected from the group consisting of a halogen atom, a hydroxyl group, a cyano group and a carbamoyl group is preferable.
More preferred is a halogen atom, a hydroxyl group, a methyl group, an ethyl group, a trifluoromethyl group, a difluoromethyl group, a hydroxymethyl group, a 2-hydroxyethyl group, a methoxy group, an ethoxy group, a trifluoromethoxy group or a difluoromethoxy group, and
particularly preferred is fluorine, chlorine or a hydroxyl group. The number of the substituents is preferably 0 to 2, more preferably 0 or 1 and most preferably 0 (unsubstituted).

Examples of the substituent of the methylene group of the “methylene group having substituent(s)” for X3 include

(1) a halogen atom;
(2) a cyano group;
(3) a hydroxyl group;
(4) a carbamoyl group;
(5) a C₁₋₃ alkyl group optionally substituted by one or more (preferably 1 to 3) substituents selected from the group consisting of a halogen atom, a hydroxyl group, a cyano group and a carbamoyl group; and
(6) a C₁₋₃ alkoxy group optionally substituted by one or more (preferably 1 to 3) substituents selected from the group consisting of a halogen atom, a hydroxyl group, a cyano group and a carbamoyl group.

The “halogen atom”, “C₁₋₃ alkyl group optionally substituted by one or more (preferably 1 to 3) substituents selected from the group consisting of a halogen atom, a hydroxyl group, a cyano group and a carbamoyl group”, and “C₁₋₃ alkoxy group optionally substituted by one or more (preferably 1 to 3) substituents selected from the group consisting of a halogen atom, a hydroxyl group, a cyano group and a carbamoyl group” mean the same as those exemplified for the aforementioned “benzene ring optionally having substituent(s)”.

As the substituent of the methylene group of the “methylene group having substituent(s)”,

(1) a halogen atom;
(2) a hydroxyl group;
(3) a C₁₋₃ alkyl group optionally substituted by one or more (preferably 1 to 3) substituents selected from the group consisting of a halogen atom, a hydroxyl group, a cyano group and a carbamoyl group; or
(4) a C₁₋₃ alkoxy group optionally substituted by one or more (preferably 1 to 3) substituents selected from the group consisting of a halogen atom, a hydroxyl group, a cyano group and a carbamoyl group is preferable.
More preferred is a fluorine atom, a chlorine atom, a bromine atom, a hydroxyl group, a methyl group, an ethyl group, a monofluoromethyl group, a difluoromethyl group, a trifluoromethyl group, a 2,2,2-trifluoroethyl group, a hydroxymethyl group, a methoxy group, an ethoxy group, a monofluoromethoxy group, a difluoromethoxy group, a trifluoromethoxy group, a 2,2,2-trifluoroethoxy group or a 2-hydroxyethoxy group, and
further preferred is a fluorine atom, a monofluoromethyl group, a difluoromethyl group or a trifluoromethyl group. The number of the substituents is preferably 1 or 2, more preferably 2.

The “imino group (—NH—) optionally having a substituent” for Y is a substituted or unsubstituted imino group. Examples of the substituent include

(1) a C₁₋₆ alkyl group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group, a carbamoyl group optionally substituted by 1 or 2 hydrocarbon groups and a thiocarbamoyl group;
(2) a C₂₋₆ alkenyl group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group, substituted by a hydrocarbon group, a sulfonyl group substituted by a hydrocarbon group, a carbamoyl group optionally substituted by 1 or 2 hydrocarbon groups and a thiocarbamoyl group;
(3) an aromatic hydrocarbon group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group, a carbamoyl group optionally substituted by 1 or 2 hydrocarbon groups and a thiocarbamoyl group; and
(4) a heterocyclic group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group, a carbamoyl group optionally substituted by 1 or 2 hydrocarbon groups and a thiocarbamoyl group.

The “halogen atom”, “hydrocarbon group”, “C₃₋₁₄ cyclic hydrocarbon group”, “heterocyclic group”, “C₁₋₆ alkyl group”, “C₂₋₆ alkenyl group” and “aromatic hydrocarbon group” mean the same as those exemplified for the “benzene ring optionally having substituent(s)”, those exemplified for the aforementioned “heterocyclic group optionally having substituent(s)”, and those exemplified for the aforementioned “hydrocarbon group optionally having substituent(s)”.

The “C₁₋₆ alkoxy group” is a straight chain or branched alkoxy group having a carbon number of 1 to 6, and a methoxy group, an ethoxy group, a propoxy group, an isopropoxy group, a butoxy group, a sec-butoxy group, a tert-butoxy group, a pentoxy group, an isopentoxy group, a neopentoxy group, a hexyloxy group and the like can be mentioned.

Examples of the “amino group optionally substituted by 1 or 2 hydrocarbon groups” include a methylamino group, a dimethylamino group, an ethylamino group, a diethylamino group, a propylamino group, an isopropylamino group, a butylamino group, a pentylamino group, a hexylamino group, a 2-propenylamino group, a 2-propynylamino group, a cyclopropylamino group, a cyclohexylamino group, a phenylamino group and the like.

Examples of the “carboxyl group optionally substituted by a hydrocarbon group” include a methoxycarbonyl group, an ethoxycarbonyl group, a propoxycarbonyl group, an isopropoxycarbonyl group, a butoxycarbonyl group, a pentoxycarbonyl group, a hexyloxycarbonyl group, an allyloxycarbonyl group, a cyclopropyloxycarbonyl group, a cyclohexyloxycarbonyl group, a phenyloxycarbonyl group and the like.

Examples of the “carbonyl group substituted by a hydrocarbon group” include an acetyl group, a propionyl group, a butyryl group, a pentanoyl group, a hexanoyl group, an acryloyl group, a propioloyl group, a benzoyl group, a 1-naphthoyl group, a 2-naphthoyl group, a cyclopropanecarbonyl group, a cyclohexanecarbonyl group and the like.

Examples of the “thio group substituted by a hydrocarbon group” include a methylthio group, an ethylthio group, a propylthio group, an isopropylthio group, a butylthio group, a pentylthio group, a hexylthio group, a 2-propenylthio group, a 2-propynylthio group, a cyclohexylthio group, a phenylthio group and the like.

Examples of the “sulfinyl group substituted by a hydrocarbon group” include a methylsulfinyl group, an ethylsulfinyl group, a propylsulfinyl group, an isopropylsulfinyl group, a butylsulfinyl group, a pentylsulfinyl group, a hexylsulfinyl group, a 2-propenylsulfinyl group, a 2-propynylsulfinyl group, a cyclohexylsulfinyl group, a phenylsulfinyl group and the like.

Examples of the “sulfonyl group substituted by a hydrocarbon group” include a methylsulfonyl group, an ethylsulfonyl group, a propylsulfonyl group, an isopropylsulfonyl group, a butylsulfonyl group, a pentylsulfonyl group, a hexylsulfonyl group, a 2-propenylsulfonyl group, a 2-propynylsulfonyl group, a cyclohexylsulfonyl group, a phenylsulfonyl group and the like.

Examples of the “carbamoyl group optionally substituted by 1 or 2 hydrocarbon groups” include a methylcarbamoyl group, a dimethylcarbamoyl group, an ethylcarbamoyl group, a diethylcarbamoyl group, a propylcarbamoyl group, an isopropylcarbamoyl group, a butylcarbamoyl group, a pentylcarbamoyl group, a hexylcarbamoyl group, a 2-propenylcarbamoyl group, a 2-propynylcarbamoyl group, a cyclopropylcarbamoyl group, a cyclohexylcarbamoyl group, a phenylcarbamoyl group and the like.

As the substituent of the imino group (—NH—) of the “imino group (—NH—) optionally having a substituent”,

(1) a C₁₋₆ alkyl group optionally substituted by one or more (preferably 1 to 3) substituents selected from the group consisting of a halogen atom, a hydroxyl group, a cyano group, a formyl group, an amino group optionally substituted by 1 or 52 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group and a carbamoyl group optionally substituted by 1 or 2 hydrocarbon groups, and a thiocarbamoyl group; or
(2) a C₂₋₆ alkenyl group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group and a carbamoyl group optionally substituted by 1 or 2 hydrocarbon groups, and a thiocarbamoyl group is preferable.
More preferred is a methyl group, an ethyl group, a 2,2,2-trifluoroethyl group, a 2-fluoroethyl group, a 2-chloroethyl group, a 2-hydroxyethyl group, a 2-dimethylaminoethyl group, a 2-diethylaminoethyl group, a methoxymethyl group, a 2-methoxyethyl group, a 2-ethoxyethyl group, a carboxymethyl group, a methoxycarbonylmethyl group, a 2-methylthioethyl group, a 2-methylsulfonylethyl group, a 2-phenylthioethyl group, a dimethylcarbamoylmethyl group or a diethylcarbamoylmethyl group, and
most preferred is a methyl group, an ethyl group, a 2-hydroxyethyl group, a 2-dimethylaminoethyl group, a carboxymethyl group or a methoxycarbonylmethyl group.

As A, a benzene ring optionally substituted by one or more substituents selected from the group consisting of

(1) a halogen atom,
(2) a hydroxyl group,
(3) a C₁₋₃ alkyl group optionally substituted by one or more (preferably 1 to 3) substituents selected from the group consisting of a halogen atom, a hydroxyl group, a cyano group and a carbamoyl group, and
(4) a C₁₋₃ alkoxy group optionally substituted by one or more (preferably 1 to 3) substituents selected from the group consisting of a halogen atom, a hydroxyl group, a cyano group and a carbamoyl group is preferable.
Preferred is a benzene ring optionally substituted by 1 to 3 substituents selected from a halogen atom, a hydroxyl group, a methyl group, an ethyl group, a monofluoromethyl group, a difluoromethyl group, a trifluoromethyl group, a 2,2,2-trifluoroethyl group, a hydroxymethyl group, a methoxy group, an ethoxy group, a monofluoromethoxy group, a difluoromethoxy group, a trifluoromethoxy group, a 2,2,2-trifluoroethoxy group and a 2-hydroxyethoxy group,
more preferred is a benzene ring optionally having a fluorine atom at the 8-position (the 8-position of 2,3-dihydro-1H-pyrrolo[3,2-c]quinoline ring), and most preferred is an unsubstituted benzene ring.

As R, a hydrogen atom, an aromatic hydrocarbon group optionally having substituent(s) or an aromatic heterocyclic group optionally having substituent(s) is preferable. More preferred is a hydrogen atom; a phenyl group optionally substituted by a C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkylcarbonyloxy group; a 3-pyrazolyl group, a 2-imidazolyl group, a 2-thienyl group, a 3-thienyl group, a 4-thiazolyl group, a 1,2,4-triazol-1-yl group, a 2-oxazolyl group or a 4-isoxazolyl group, each of which is optionally substituted by a C₁₋₃ alkyl group optionally substituted by one or more (preferably 1 to 3) substituents selected from the group consisting of a halogen atom, a hydroxyl group, a cyano group, a carbamoyl group, a C₁₋₃ alkoxy group and a C₁₋₆ alkyl-carbonyloxy group; or an unsubstituted phenyl, further preferred is a hydrogen atom, a phenyl group, a 2-thienyl group, a 3-thienyl group, a 3-pyrazolyl group, a 2-imidazolyl group or a 1-(pivaloyloxymethyl)-2-imidazolyl group, and most preferred is a phenyl group, a 3-thienyl group or a 2-imidazolyl group.

As Z, an aromatic hydrocarbon group optionally having substituent(s) or an aromatic heterocyclic group optionally having substituent(s) is preferable.

More preferred is an unsubstituted phenyl group, a phenyl group substituted by a halogen atom, a phenyl group substituted by a C₁₋₃ alkyl group optionally substituted by a halogen atom, a phenyl group substituted by a C₁₋₃ alkoxy group optionally substituted by a halogen atom, a phenyl group substituted by a phenyl group optionally substituted by a halogen atom, or a phenyl group having a 1-pyrazolyl group, a 2-imidazolyl group, a 2-thienyl group, a 3-thienyl group, a 4-thiazolyl group, a 1,2,4-triazol-1-yl group, a 2-oxazolyl group, a 5-oxazolyl group or a 4-isoxazolyl group, each of which is optionally substituted by one or more (preferably 1 to 3) substituents selected from the group consisting of a halogen atom, a hydroxyl group, a cyano group, a carbamoyl group, a C₁₋₃ alkyl group and a C₁₋₃ alkoxy group, further preferred is a phenyl group, a 4-fluorophenyl group, a 3-fluorophenyl group, a 2-fluorophenyl group, a 4-methylphenyl group, a 4-methoxyphenyl group, a 4-trifluoromethoxyphenyl group, a 4-difluoromethoxyphenyl group, a 4-biphenylyl group, a 4-(3-methyl-1-pyrazolyl)phenyl group, a 4-(1-pyrazolyl)phenyl group, a 4-(2-methyl-4-thiazolyl)phenyl group, a 4-(1,2,4-triazol-1-yl)phenyl group, a 4-(2-oxazolyl)phenyl group, a 4-(5-oxazolyl)phenyl group, a 4-(2-methyl-5-oxazolyl)phenyl group, or a 4-(3-methyl-4-isoxazolyl)phenyl group, and most preferred is a 4-(3-methyl-1-pyrazolyl)phenyl group or a 4-(1-pyrazolyl)phenyl group.

As X1 and X2, a divalent C₁₋₅ chain hydrocarbon group optionally substituted by one or more (preferably 1 to 3) substituents selected from the group consisting of

(1) a halogen atom,
(2) a hydroxyl group,
(3) a C₁₋₃ alkyl group optionally substituted by one or more (preferably 1 to 3) substituents selected from the group consisting of a halogen atom and a hydroxyl group, and
(4) a C₁₋₃ alkoxy group optionally substituted by one or more (preferably 1 to 3) substituents selected from the group consisting of a halogen atom and a hydroxyl group is preferable.
More preferred is a divalent C₁₋₅ chain hydrocarbon group optionally having 0 to 2 substituents selected from a halogen atom, a hydroxyl group, a methyl group, an ethyl group, a trifluoromethyl group, a difluoromethyl group, a hydroxymethyl group, a 2-hydroxyethyl group, a methoxy group, an ethoxy group, a trifluoromethoxy group and a difluoromethoxy group, further preferred is an unsubstituted divalent C₁₋₅ chain hydrocarbon group, and most preferred is an embodiment wherein one is methylene (—CH₂—) and the other is ethylene (—(CH₂)₂—)—).

As X3, a methylene group substituted by 1 or 2 substituents selected from the group consisting of

(1) a halogen atom,
(2) a hydroxyl group,
(3) a C₁₋₃ alkyl group optionally substituted by one or more (preferably 1 to 3) substituents selected from the group consisting of a halogen atom and a hydroxyl group, and
(4) a C₁₋₅ alkoxy group optionally substituted by one or more (preferably 1 to 3) substituents selected from the group consisting of a halogen atom and a hydroxyl group is preferable.
More preferred is a methylene group substituted by 1 or 2 substituents selected from a halogen atom, a hydroxyl group, a methyl group, an ethyl group, a trifluoromethyl group, a difluoromethyl group, a hydroxymethyl group, a 2-hydroxyethyl group, a methoxy group, an ethoxy group, a trifluoromethoxy group and a difluoromethoxy group, further preferred is difluoromethylene (—CF₂—), fluoromethylene (—CHF—), bis(trifluoromethyl)methylene (—C(CF₃)₂—) or (trifluoromethyl)methylene (—CH(CF₃)—), and most preferred is difluoromethylene (—CF₂—).

As Y, a bond; an (unsubstituted) imino group; a C₁₋₆ alkylimino group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group, a carbamoyl group optionally substituted by 1 or 2 hydrocarbon groups and a thiocarbamoyl group; or a C₂₋₆ alkenylimino group optionally substituted by one or more (preferably 1 to 3) substituents selected from the group consisting of a halogen atom, a hydroxyl group, a cyano group, a formyl group, an amino group optionally substituted by 1 or 2 hydrocarbon groups, a C₃₋₁₄ cyclic hydrocarbon group, a heterocyclic group, a C₁₋₆ alkoxy group, a carboxyl group optionally substituted by a hydrocarbon group, a carbonyl group substituted by a hydrocarbon group, a thio group substituted by a hydrocarbon group, a sulfinyl group substituted by a hydrocarbon group, a sulfonyl group substituted by a hydrocarbon group, a carbamoyl group optionally substituted by 1 or 2 hydrocarbon groups and a thiocarbamoyl group is preferable.

More preferred is a bond, an (unsubstituted) imino group, a C₁₋₆ alkylimino group optionally substituted by a halogen atom, a C₁₋₆ alkylimino group optionally substituted by a hydroxyl group, a C₁₋₆ alkylimino group optionally substituted by an amino group optionally substituted by 1 or 2 C₁₋₃ alkyl groups, a C₁₋₆ alkylimino group optionally substituted by a C₁₋₆ alkoxy group, or a C₁₋₆ alkylimino group optionally substituted by a carboxyl group optionally substituted by a C₁₋₃ alkyl group, further preferred is a bond, an (unsubstituted) imino group, a methylimino group, an ethylimino group, a 2-hydroxyethylimino group, a 2-dimethylaminoethylimino group, a carboxymethylimino group, or a methoxycarbonylmethylimino group, and most preferred is a bond or an (unsubstituted) imino group.

When A is an unsubstituted benzene ring or a benzene ring substituted by a halogen atom, R is preferably an aromatic hydrocarbon group or an aromatic heterocyclic group each optionally having substituent(s), Z is preferably a phenyl group having a 1-pyrazolyl group, a 2-imidazolyl group, a 2-thienyl group, a 3-thienyl group, a 4-thiazolyl group, a 1,2,4-triazol-1-yl group, a 2-oxazolyl group, a 5-oxazolyl group or a 4-isoxazolyl group, each of which is optionally substituted by one or more (preferably 1 to 3) substituents selected from the group consisting of a halogen atom, a hydroxyl group, a cyano group, a carbamoyl group, a C₁₋₃ alkyl group and a C₁₋₃ alkoxy group, X1 and X2 are preferably an unsubstituted divalent C₁₋₅ chain hydrocarbon group, and X3 is preferably a methylene group substituted by 1 or 2 substituents selected from a halogen atom, a hydroxyl group, a methyl group, an ethyl group, a trifluoromethyl group, a difluoromethyl group, a hydroxymethyl group, a 2-hydroxyethyl group, a methoxy group, an ethoxy group, a trifluoromethoxy group and a difluoromethoxy group. As Y, a bond, an (unsubstituted) imino group, a C₁₋₆ alkylimino group optionally substituted by a halogen atom, a C₁₋₆ alkylimino group optionally substituted by a hydroxyl group, a C₁₋₆ alkylimino group optionally substituted by an amino group optionally substituted by 1 or 2 C₁₋₃ alkyl groups, a C₁₋₆ alkylimino group optionally substituted by a C₁₋₆ alkoxy group, or a C₁₋₆ alkylimino group, optionally substituted by a carboxyl group optionally substituted by a C₁₋₃ alkyl group is preferable.

As compound (I), the following compounds are preferable.

### [Compound A]

A compound of the formula (I) wherein

A is a benzene ring optionally substituted by fluorine atom(s),
R is an aromatic hydrocarbon group optionally having substituent(s) or an aromatic heterocyclic group optionally having substituent(s),
X1 is ethylene (—CH₂CH₂—),
X2 is methylene (—CH₂—),
X3 is a methylene group having substituents selected from (1) a fluorine atom and (2) a C₁₋₃ alkyl group substituted by fluorine atom(s),
Y is a bond or an imino group (—NH—), and
Z is an aromatic hydrocarbon group optionally having substituent(s) or an aromatic heterocyclic group optionally having substituent(s), or a salt thereof.

### [Compound B]

A compound of the formula (I) wherein

A is a benzene ring optionally substituted by fluorine atom(s),
R is (1) a phenyl group,

(2) a thienyl group (e.g., a 3-thienyl group) or

(3) an imidazolyl group (e.g., 2-imidazolyl group) optionally substituted by a C₁₋₃ alkyl group optionally substituted by a C₁₋₆ alkyl-carbonyloxy group,

X1 is ethylene (—CH₂CH₂—),
X2 is methylene (—CH₂—),
X3 is a methylene group having fluorine atom(s) (e.g., difluoromethylene (—CF₂—)),
Y is a bond, and
Z is a phenyl group having a pyrazolyl group (e.g., a 1-pyrazolyl group) optionally substituted by a C₁₋₃ alkyl group, or a salt thereof.

As compound (I), the following compounds are more preferable.
N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide
N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrobromide
N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrochloride
N-[(1R,2S)-4,4-difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide
N-[(1R,2S)-4,4-difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrochloride
N-{(1R,2S)-4,4-difluoro-2-[(4-thiophen-3-yl-2,3-dihydro-1H-pyrrolo[3,2-c]quinolin-1-yl)carbonyl]cyclohexyl}-4-(3-methyl-1H-pyrazol-1-yl)benzamide
N-{(1R,2S)-4,4-difluoro-2-[(4-thiophen-3-yl-2,3-dihydro-1H-pyrrolo[3,2-c]quinolin-1-yl)carbonyl]cyclohexyl}-4-(3-methyl-1H-pyrazol-1-yl)benzamide hydrochloride
N-{(1R,2S)-4,4-difluoro-2-[(8-fluoro-4-phenyl-2,3-dihydro-1H-pyrrolo[3,2-c]quinolin-1-yl)carbonyl]cyclohexyl}-4-(3-methyl-1H-pyrazol-1-yl)benzamide
N-{(1R,2S)-4,4-difluoro-2-[(8-fluoro-4-phenyl-2,3-dihydro-1H-pyrrolo[3,2-c]quinolin-1-yl)carbonyl]cyclohexyl}-4-(3-methyl-1H-pyrazol-1-yl)benzamide hydrochloride

The compound of the present invention sometimes contains an isomer due to the structure, and such optically active isomer or stereoisomer and a mixture thereof are also encompassed in the present invention.

In addition, when the compound of the present invention forms a salt, or a hydrate and/or a solvate, they are also encompassed in the present invention.

The salt of the compound of the present invention is a pharmaceutically acceptable salt. When the compound has an acidic functional group therein, salts such as inorganic salts such as alkali metal salts (sodium salt, potassium salt etc.), alkaline earth metal salts (calcium salt, magnesium salt, barium salt etc.) and the like, ammonium salt and the like can be mentioned, and when the compound has a basic functional group therein, for example, salts with inorganic acid such as hydrochloric acid, hydrobromic acid, nitric acid, sulfuric acid, phosphoric acid and the like, and salts with organic acid such as acetic acid, phthalic acid, fumaric acid, oxalic acid, tartaric acid, maleic acid, citric acid, succinic acid, methanesulfonic acid, p-toluenesulfonic acid and the like can be mentioned.

The hydrate and/or solvate may be any of hydrate, solvate and a mixture thereof.

Compound (I) may be a crystal, and both a single crystal and a crystal mixture are encompassed in the compound of the present invention. A compound labeled with an isotope (e.g., ³H, ¹¹C, ¹⁴C, ¹⁸F, ³⁵S, ¹²⁵I etc.) and the like and a deuterated compound are also encompassed in the compound of the present invention.

Compound (I) of the present invention can be synthesized by, for example, the method shown in the following reaction scheme 1.

Each compound in the reaction scheme also shows an enantiomer of the compound having the structural formula described therein, including the compound of the present invention, though the description is omitted. Each compound in the reaction scheme may form a salt. Examples of such salt include metal salt, ammonium salt, salt with organic base, salt with inorganic acid, salt with organic acid, salt with basic or acidic amino acid and the like. Examples of the metal salt include alkali metal salts such as sodium salt, potassium salt and the like; alkaline earth metal salts such as calcium salt, magnesium salt, barium salt and the like; aluminum salt and the like. Examples of the salt with organic base include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine and the like. Examples of the salt with inorganic acid include salts with hydrochloric acid, hydrobromic acid, nitric acid, sulfuric acid, phosphoric acid and the like. Examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like. Examples of the salt with basic amino acid include salts with arginine, lysine, ornithine and the like, and examples of the salt with acidic amino acid include salts with aspartic acid, glutamic acid and the like. Of these, pharmaceutically acceptable salts are preferable.

In addition, each compound in the reaction scheme may be any of hydrate and/or solvate. Examples of the hydrate and the like include 0.23 hydrate, 0.5 hydrate, 1 hydrate, 1 hydrate 1 solvate, 2 solvate and the like.

A reaction mixture of the compound obtained in each step can be directly used as a crude product for the next reaction. It can also be isolated from the reaction mixture according to a conventional method, and can be easily purified by a known method per se, for example, separation means such as extraction, concentration, neutralization, filtration, distillation, recrystallization, chromatography and the like. Alternatively, when the compound in the reaction scheme is commercially available, the commercially available product can also be used directly.

Schematic reaction formulas are shown below. In the schematic reaction formulas, P (P1, P2, P3) show a protecting group or a hydrogen atom. R1 and R2 are each a C₁₋₆ alkyl group such as a methyl group and the like. Other symbols are each as defined above.

As a protecting group for P, the groups generally used for peptide chemistry and the like can be mentioned. The groups described in Protective Groups in Organic Synthesis, 3rd Ed. (1999), authored by Theodora W. Greene, Peter G. M. Wuts, published by Wiley-Interscience and the like can be mentioned. As P1 and P2, a tert-butoxycarbonyl group, a benzyloxycarbonyl group and the like can be specifically mentioned. As P3, an ethyl group, a methyl group and the like can be specifically mentioned.

In any step, when desired, one or more of known deprotection, acylation reaction, alkylation reaction, hydrogenation reaction, oxidation reaction, reduction reaction, carbon chain extension reaction or substituent exchange reaction may be further combined to synthesize the compound of each step.

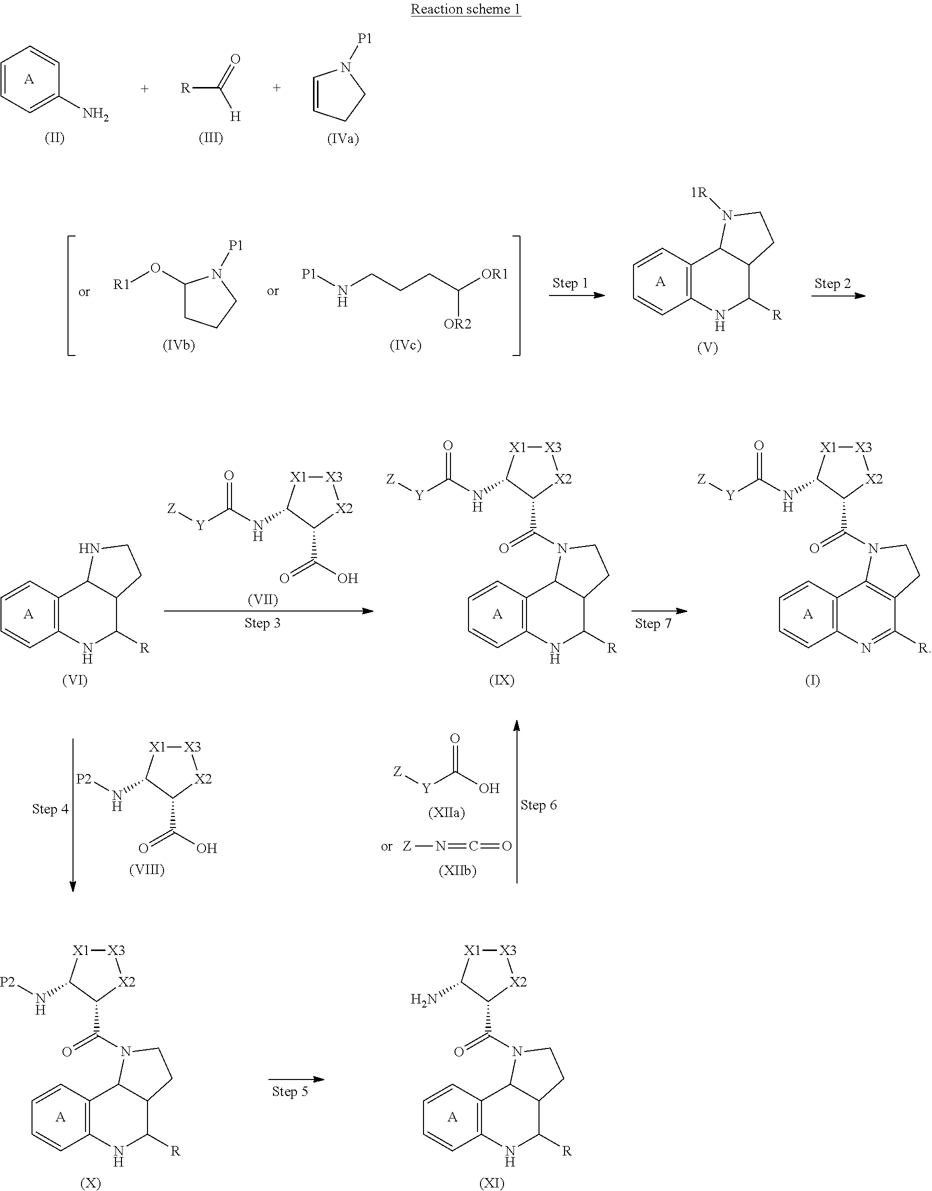

By three components condensation reaction of compound (II), compound (III) and compound (IVa), compound (V) is synthesized (step 1), the protecting group of compound (V) is removed to give compound (VI) (step 2), and the amino group of compound (VI) is acylated with compound (VII) (step 3), whereby compound (IX) can be produced. Compound (IVb) or compound (IVc) may be used instead of compound (IVa). Alternatively, compound (VI) is acylated with compound (VIII) to give compound (X) (step 4), the protecting group of compound (X) is removed to give compound (XI) (step 5), and amine of compound (XI) is acylated with compound (XIIa) (step 6), whereby compound (IX) can be produced. When Y is an imino group (—NH—), compound (XIIb) may be used instead of compound (XIIa). Compound (I) can be produced from compound (IX) by an oxidation reaction (step 7).

### (Step 1)

In step 1, compound (V) is synthesized by 3 components condensation reaction of three compounds of compound (II), compound (III) and compound (IVa). This reaction can be performed in the presence of a catalyst.

The amount of compound (III) to be used is about 1 mol to 2 mol, preferably about 1 mol to 1.2 mol, relative to 1 mol of compound (II). The amount of compound (IVa) to be used is about 1 mol to 2 mol, preferably about 1 mol to 1.2 mol, relative to 1 mol of compound (II). The amount of the catalyst to be used is about 0.01 mol to 2 mol, preferably about 0.1 mol to 1 mol, relative to 1 mol of compound (II). As the catalyst, protonic acid (acetic acid, trifluoroacetic acid, 3,4-dihydroxy-3-cyclobutene-1,2-dione, hydrochloric acid, sulfuric acid, phosphoric acid etc.), Lewis acid (BF₃.Et₂O, AlCl₃, InCl₃, TiCl₄, ZrCl₄, HfCl₄, Cu (OTf)₂, Zn(OTf)₂, Sc(OTf)₃, Y(OTf)₃, La(OTf)₃, Eu(OTf)₃, Dy(OTf)₃, Yb(OTf)₃ etc.) and the like can be used. As necessary, a dehydrating agent such as anhydrous magnesium sulfate, molecular sieves and the like may be added. The amount of the dehydrating agent to be used is about 1 to 20 mol, preferably about 1 to 10 mol, relative to 1 mol of compound (II).

While the solvent for this reaction is not particularly limited as long as the reaction proceeds, acetonitrile, toluene and the like are preferable. The reaction temperature is generally 0° C. to 100° C., preferably 10° C. to 40° C. The reaction time is generally 1 hr to 100 hr, preferably 1 hr to 24 hr. In this reaction, a stereoisomers mixture of compound (V) in an endo-form and compound (V) in an exo-form is often obtained.

### (Step 2)

In step 2, the protecting group of compound (V) is removed to synthesize compound (VI). This reaction varies depending on the kind of protecting group P1 and, for example, the method described in Protective Groups in Organic Synthesis, 3rd Ed. (1999), authored by Theodora W. Greene, Peter G. M. Wuts, published by Wiley-Interscience can be employed.

When the protecting group P1 is a tert-butoxycarbonyl group, for example, a solution of hydrochloric acid in ethyl acetate or trifluoroacetic acid is used as a reaction agent. Where necessary, methanol, ethanol, tetrahydrofuran, acetonitrile or ethyl acetate may be added as a solvent. The reaction temperature is generally 0° C. to 100° C., preferably 10° C. to 40° C. The reaction time is generally 1 hr to 100 hr, preferably 1 hr to 24 hr.

When the protecting group P1 is a benzyloxycarbonyl group, for example, palladium carbon is used as a reaction agent under a hydrogen atmosphere. While the solvent of this reaction is not particularly limited as long as the reaction proceeds, methanol or ethanol is preferable. The reaction temperature is generally 0° C. to 60° C., preferably 10° C. to 40° C. The reaction time is generally 1 hr to 100 hr, preferably 1 hr to 24 hr.

### (Step 3)

In step 3, the amino group of compound (VI) is acylated with compound (VII) in the presence of a suitable condensing agent and, where necessary, a base to synthesize compound (IX).

The amount of compound (VII) to be used is about 1 mol to 2 mol, preferably about 1 mol to 1.2 mol, relative to 1 mol of compound (VI). The amount of the condensing agent to be used is about 1 mol to 10 mol, preferably about 1 mol to 1.2 mol, relative to 1 mol of compound (VI). Example of the condensing agent include carbodiimides (DCC (i.e., 1,3-dicyclohexylcarbodiimide), WSC (i.e., 1-ethyl-3-(3-dimethylaminopropyl)carbodiimide hydrochloride), DIC (i.e., 2-dimethylaminoisopropyl chloride hydrochloride) etc.), phosphoric acid derivative (diethyl cyanophosphate, diphenylphosphoryl azide, BOP-Cl (i.e., bis(2-oxo-3-oxazolidinyl)phosphinic chloride) etc.) and the like. These can be used alone or in combination with additives (e.g., N-hydroxysuccinimide, 1-hydroxybenzotriazole, 3-hydroxy-4-oxo-3,4-dihydro-1,2,3-benzotriazine etc.). The amount of the additive to be used is about 1 to 2 mol, preferably about 0.05 to 1.2 mol, relative to 1 mol of compound (VI). As the base, inorganic bases such as sodium hydride, sodium hydroxide, potassium hydroxide and the like, aromatic amines such as pyridine, lutidine and the like, tertiary amines such as triethylamine, tripropylamine, tributylamine, cyclohexyldimethylamine, 4-dimethylaminopyridine, N,N-dimethylaniline, N-methylpiperidine, N-methylpyrrolidine, N-methylmorpholine and the like, ammonia or a mixture of two or more kinds of these and the like are used.

While the solvent for this reaction is not particularly limited as long as the reaction proceeds, acetonitrile, toluene, tetrahydrofuran, N,N-dimethylformamide and the like are preferable, and these solvents may be used in a mixture. The reaction temperature is generally 0° C. to 100° C., preferably 10° C. to 40° C. The reaction time is generally 1 hr to 100 hr, preferably 1 hr to 24 hr.

In addition, compound (IX) can also be obtained by reacting compound (VI) with a reactive derivative (acid halide, acid anhydride, active ester, ester, acid imidazolide, acid azide etc.) of compound (VII) according to a known method.

### (Step 4)

In step 4, the amino group of compound (VI) is acylated with compound (VIII) to synthesize compound (X), and a method similar to that of step 3 is used.

### (Step 5)

In step 5, the protecting group of compound (X) is removed to synthesize compound (XI), and a method similar to that of step 2 is used.

### (Step 6)

In step 6, the amino group of compound (XI) is acylated with compound (XIIa) or compound (XIIb) to synthesize compound (IX).

When compound (XIIa) is used, a method similar to that of step 3 is used.

When compound (XIIb) is used, the reaction is performed in the presence of a base as necessary. The amount of compound (XIIb) to be used is about 0.1 mol to 2 mol, preferably about 1 mol to 1.2 mol, relative to 1 mol of compound (XI). As the base, inorganic bases such as sodium hydride, sodium hydroxide, potassium hydroxide and the like, aromatic amines such as pyridine, lutidine and the like, tertiary amines such as triethylamine, tripropylamine, tributylamine, cyclohexyldimethylamine, 4-dimethylaminopyridine, N,N-dimethylaniline, N-methylpiperidine, N-methylpyrrolidine, N-methylmorpholine and the like, ammonia or a mixture of two or more kinds of these and the like are used.

While the solvent for this reaction is not particularly limited as long as the reaction proceeds, acetonitrile, toluene, tetrahydrofuran, N,N-dimethylformamide and the like are preferably used, and these solvents may be used in a mixture. The reaction temperature is generally 0° C. to 100° C., preferably 10° C. to 40° C. The reaction time is generally 1 hr to 100 hr, preferably 1 hr to 24 hr.

As the synthesis method of compound (XIIb), the synthesis method of isocyanate described in Jikken Kagaku Koza (Courses in Experimental Chemistry), 5th Ed., vol. 14 (2005, Maruzen Press), Jikken Kagaku Koza (Courses in Experimental Chemistry), 4th Ed., vol. 20 (1992, Maruzen Press) etc. and the like can be adopted.

### (Step 7)

In step 7, compound (IX) is treated with a suitable oxidizing agent to allow conversion into the corresponding compound (I).

The amount of the oxidizing agent to be used is about 1 mol to 100 mol, preferably about 1 mol to 80 mol, relative to 1 mol of compound (IX). Examples of the oxidizing agent include manganese dioxide, quinone oxidizing agent [DDQ (i.e., 2,3-dichloro-5,6-dicyano-1,4-benzoquinone), chloranil (2,3,5,6-tetrachloro-1,4-benzoquinone) etc.] and the like.

While the solvent for this reaction is not particularly limited as long as the reaction proceeds, acetonitrile, tetrahydrofuran, toluene and the like are preferable. The reaction temperature is generally 0° C. to 100° C., preferably 10° C. to 80° C. The reaction time is generally 1 hr to 100 hr, preferably 1 hr to 24 hr.

Now, the synthesis methods of compound (VII) and compound (VIII) necessary for synthesizing compound (I) are shown in the following reaction scheme 2.

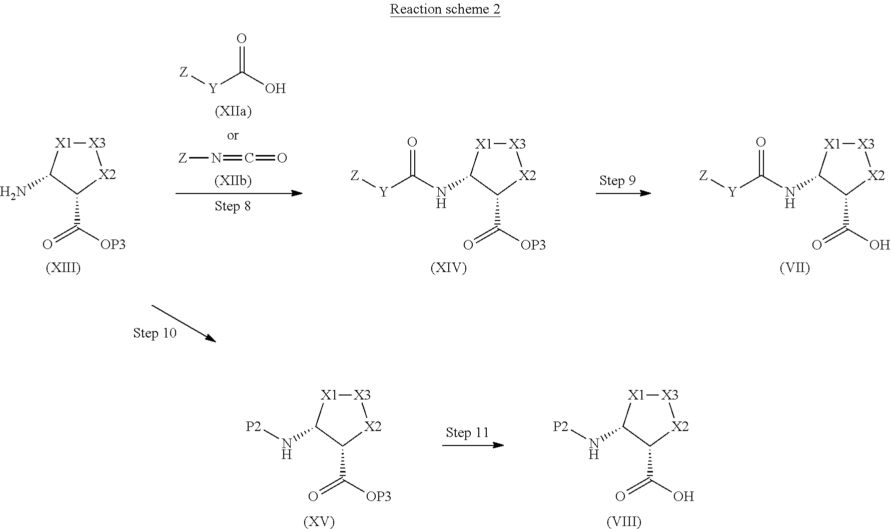

Compound (VII) can be synthesized by acylating compound (XIII) with compound (XIIa) or (XIIb) to give compound (XIV) (step 8) and hydrolyzing the ester of compound (XIV) (step 9). Compound (VIII) can be synthesized by protecting the amino group of compound (XIII) to give compound (XV) (step 10) and hydrolyzing the ester of compound (XV) (step 11).

### (Step 8)

In step 8, compound (XIII) is acylated with compound (XIIa) or (XIIb) to synthesize compound (XIV), and a method similar to that of step 6 is used.

Compound (XIII) can be synthesized according to, for example, the method described in WO2008/153027.

### (Step 9)

In step 9, compound (XIV) is hydrolyzed under basic conditions to synthesize compound (VII). As the base, sodium hydroxide, potassium hydroxide, lithium hydroxide and the like can be used.

While the solvent for this reaction is not particularly limited as long as the reaction proceeds, tetrahydrofuran, ethanol, methanol, water and the like are preferable, and they may be used in a mixture. The reaction temperature is generally −10° C. to 100° C., preferably 0° C. to 80° C. The reaction time is generally 1 hr to 100 hr, preferably 1 hr to 24 hr.

### (Step 10)

In step 10, the amino group of compound (XIII) is protected to synthesize compound (XV).

The reaction varies depending on the kind of protecting group P2 and, for example, the method described in Protective Groups in Organic Synthesis, 3rd Ed. (1999), authored by Theodora W. Greene, Peter G. M. Wuts, published by Wiley-Interscience, can be used.

When protecting group P2 is a tert-butoxycarbonyl group, di-tert-butyl dicarbonate and the like are used as the reaction agent. As the base, sodium hydroxide, potassium hydroxide, sodium hydrogen carbonate, potassium hydrogen carbonate, sodium carbonate, potassium carbonate and the like are used. While the solvent for this reaction is not particularly limited as long as the reaction proceeds, water, tetrahydrofuran and the like are preferable, and two or more kinds of solvents may be used in a mixture. The reaction temperature is generally 0° C. to 100° C., preferably 10° C. to 40° C. The reaction time is generally 1 hr to 100 hr, preferably 1 hr to 24 hr.

When protecting group P2 is a benzyloxycarbonyl group, benzyl chlorocarbonate is used as a reaction agent. As the base, sodium hydroxide, potassium hydroxide, sodium hydrogen carbonate, potassium hydrogen carbonate, sodium carbonate, potassium carbonate and the like are used. While the solvent for this reaction is not particularly limited as long as the reaction proceeds, water, tetrahydrofuran and the like are preferable, and two or more kinds of these solvents may be used in a mixture. The reaction temperature is generally 0° C. to 100° C., preferably 10° C. to 40° C. The reaction time is generally 1 hr to 100 hr, preferably 1 hr to 24 hr.

### (Step 11)

In step 11, compound (XV) is hydrolyzed to synthesize compound (VIII), and a method similar to that of step 9 is used.

Compound (I) may be used as a prodrug. The prodrug of compound (I) means a compound which is converted to compound (I) under physiological conditions in vivo, as a result of a reaction with an enzyme, gastric acid etc. Thus, the compound is converted into compound (I) by enzymatical oxidation, reduction, hydrolysis or the like, or by hydrolysis due to gastric acid or the like, etc.

As a prodrug of compound (I), a compound obtained by subjecting an amino group of compound (I) to an acylation, alkylation or phosphorylation (e.g., a compound obtained by subjecting an amino group of compound (I) or a nitrogen atom contained in a heterocyclic group to an eicosanoylation, alanylation, pentylaminocarbonylation, (5-methyl-2-oxo-1,3-dioxolen-4-yl)methoxycarbonylation, tetrahydrofuranylation, pyrrolidylmethylation, pivaloyloxymethylation, tert-butylation, etc.); a compound obtained by subjecting a hydroxyl group in compound (I) to an acylation, alkylation, phosphorylation and boration (e.g., a compound obtained by subjecting a hydroxyl group of compound (I) to an acetylation, palmitoylation, propanoylation, pivaloylation, succinylation, fumarylation, alanylation, dimethylaminomethylcarbonylation, etc.); a compound obtained by subjecting a carboxy group of compound (I) to an esterification or amidation (e.g., a compound obtained by subjecting a carboxy group of compound (I) to an ethyl-esterification, phenyl-esterification, carboxymethyl-esterification, dimethylaminomethyl-esterification, pivaloyloxymethyl-esterification, ethoxycarbonyloxyethyl-esterification, phthalidylesterification, (5-methyl-2-oxo-1,3-dioxolen-4-yl)methyl-esterification, cyclohexyloxycarbonylethyl-esterification, methylamidation, etc.) and the like can be mentioned. These compounds can be produced from compound (I) by a known method. In addition, the prodrug of compound (I) may be a compound, which is converted to compound (I) under the physiological conditions, as described in Pharmaceutical Research and Development, vol. 7 (Drug Design), pp. 163-198 (1990), published by Hirokawa Publishing Co.

Compound (I) is safe and lower toxic (e.g., is low in acute toxicity, chronic toxicity, genetic toxicity, reproductive toxicity, cardiotoxicity, drug interaction, and carcinogenicity), is superior in the physical property (e.g., solubility, membrane permeability, metabolic stability, thermal stability) and pharmacokinetics, and can be used as an NK receptor antagonist exhibiting high NK2 selectivity and advantageous effect, particularly an NK2 receptor antagonist.

The drug interaction is a phenomenon of decreased or increased plasma concentration of concomitant drugs or the drug itself. Induction of drug-metabolizing enzyme such as CYP is known to cause efficacy attenuation since it decreases plasma concentration of concomitant drugs or the drug itself (Lin J. H., CYP induction-mediated drug interactions: in vitro assessment and clinical implications. Pharm Res 23: 1089-1116, 2006).

On the other hand, inhibition of drug-metabolizing enzyme increases plasma concentration of concomitant drugs and/or the drug itself and causes side effects due to the concomitant drugs and/or the drug itself. Of the drug-metabolizing enzyme inhibitions, what is called time-dependent inhibition (TDI) is a phenomenon showing maintained inhibitory action even after disappearance of the inhibitor from the body, and is known to highly increase the plasma concentration of concomitant drugs and the drug itself in a sustained manner (Venkatakrishnan K. et al., Mechanism-based inactivation of human cytochrome P450 enzymes: strategies for diagnosis and drug-drug interaction risk assessment. Xenobiotica 37: 1225-1256, 2007).

Of the drug-metabolizing enzymes, since CYP3A is involved in 50% of the metabolism of pharmaceutical products, the drug interaction based on CYP3A induction or TDI gives a great influence, and therefore, study of CYP3A induction and TDI of the compound is an important factor in drug discovery. Since compound (I) is low in CYP3A-inducing activity and TDI activity, the possibility of inducing drug interaction is considered to be low.

Compound (I) having a superior NK2 receptor antagonistic action is useful as an agent for the prophylaxis and/or treatment of diseases such as inflammation or allergic diseases (atopy, dermatitis, herpes, psoriasis, asthma, bronchitis, chronic obstructive pulmonary disease, sputum, rhinitis, rheumatoid arthritis, osteoarthritis, osteoporosis, multiple sclerosis, conjunctivitis, cystitis and the like), pain, migraine, neuralgic pain, pruritus, cough, and further the diseases in the central nervous system [schizophrenia, Parkinsonism, melancholia, anxiety neurosis, compulsive neurosis, panic disorder, dementia (Alzheimer's disease and the like) and the like], gastrointestinal diseases [functional gastrointestinal diseases (irritable bowel syndrome, nonulcer dyspepsia, functional dyspepsia and the like), ulcerative colitis, Crohn's disease, abnormalities caused by urease positive herical gram negative bacteria (Helicobacter pylori and the like) (gastritis, gastric ulcer and the like) and the like], vomiting, abnormal urination (pollakiuria, incontinence of urine, and the like), circulartory diseases (angina pectoris, hypertension, cardiac failure, thrombosis and the like), immune abnormality, cancer, HIV infection, cardiovascular diseases, solar dermatitis, sexual inadequacy, ataxia, dysgnosia or circadian rhythm disorder and the like in mammals (mice, rats, hamsters, rabbits, cats, dogs, bovines, sheep, monkeys, humans and the like). Of these, it is useful as an agent for the prophylaxis and/or treatment of functional gastrointestinal diseases (irritable bowel syndrome, nonulcer dyspepsia, functional dyspepsia and the like).

A medicament containing the compound of the present invention is obtained using the compound of the present invention alone or along with a pharmacologically acceptable carrier according to a method known per se as a production method of pharmaceutical preparations (e.g., the method described in the Japanese Pharmacopoeia etc.) and can be administered safely as, for example, tablet (including sugar-coated tablet, film-coated tablet, sublingual tablet, orally disintegrable tablet, buccal tablet and the like), pill, powder, granule, capsule (including soft capsule, microcapsule), troche, syrup, liquid, emulsion, suspension, controlled release preparation (e.g., immediate-release preparation, sustained-release preparation, sustained-release microcapsule), aerosol, films (e.g., orally disintegrable films, oral mucosal adhesive film), injection (e.g., subcutaneous injection, intravenous injection, intramuscular injection, intraperitoneal injection), drip infusion, transdermal absorption type preparation, ointment, lotion, adhesive preparation, suppository (e.g., rectal suppository, vaginal suppository), pellet, nasal preparation, pulmonary preparation (inhalant), eye drop and the like, orally or parenterally (e.g., intravenous, intramuscular, subcutaneous, intraorgan, intranasal, intradermal, instillation, intracerebral, rectal, vaginal, intraperitoneal, intratumor, tumor proximal administration and the like and direct administration to a lesion).

As pharmacologically acceptable carrier, various organic or inorganic carriers conventionally used as starting materials of preparations are used, which are added as excipient, lubricant, binder and disintegrant for solid preparations; solvent, solubilizing agent, suspending agent, isotonicity agent, buffer and soothing agent for liquid preparations; and the like. Where necessary, additives for preparations such as preservative, antioxidizing agent, colorant, sweetener and the like can be also used.

While the pharmaceutical composition varies depending on the dosage form, the administration method, carrier and the like, the composition can be produced by adding the compound of the present invention in a proportion of generally 0.01-100% (w/w), preferably 0.1-95% (w/w), of the total amount of the preparation according to a conventional method.

While the dose of the compound (I) varies depending on the administration route, symptoms and the like, it is, for example, 0.01-1000 mg/kg body weight/day, preferably 0.01-100% mg/kg body weight/day, more preferably 0.5-100 mg/kg body weight/day, particularly preferably 0.1-10 mg/kg body weight/day, further preferably 1-50 mg/kg body weight/day, particularly preferably 1-25 mg/kg body weight/day, for, for example, oral administration to patients with irritable bowel syndrome (adult, body weight 40 to 80 kg: e.g., 60 kg). This amount can be administered once a day or in 2 or 3 portions a day.

In addition, the compound of the present invention can be used in combination with other active ingredients (hereinafter to be abbreviated as concomitant drug).

While the compound of the present invention shows a superior NK receptor antagonistic activity, particularly NK2 receptor antagonistic activity, when used as a single agent, its effect can be still more enhanced by the use together with one or more of the concomitant drugs (combined use of multi-agents).

As the concomitant drug, for example, the following can be mentioned.

### (1) Therapeutic Agents for Diabetes

Insulin preparations (animal insulin preparations extracted from the bovine or swine pancreas; human insulin preparations synthesized by a genetic engineering technique using Escherichia coli or a yeast; insulin zinc; protamine zinc insulin; fragment or derivative of insulin etc.), insulin sensitizers (pioglitazone hydrochloride, troglitazone, rosiglitazone or a maleate thereof etc.), α-glucosidase inhibitors (voglibose, acarbose, miglitol etc.), biguanides (phenformin, metformin, buformin etc.), sulfonylureas (tolbutamide, glibenclamide, gliclazide, chlorpropamide, tolazamide, acetohexamide, glyclopyramide, glimepiride etc.), other insulin secretagogues (repaglinide, senaglinide, mitiglinide or a calcium salt hydrate thereof, nateglinide etc.), GLP-1 agonists (liraglutide etc.), dipeptidyl peptidase IV inhibitors (sitagliptin, vildagliptin, saxagliptin, alogliptin etc.), β3 agonists ((2R)-6-[(2R)-2-{[(2R)-2-(3-chlorophenyl)-2-hydroxyethyl]amino}propyl]-2,3-dihydro-1,4-benzodioxin-2-carboxylic acid etc.), gluconeogenesis inhibitors (glycogen phosphorylase inhibitors, glucose-6-phosphatase inhibitors, glucagon antagonists etc.) and the like.

### (2) Therapeutic Agents for Diabetic Complications

Aldose reductase inhibitors (ranirestat, epalrestat, lidorestat, fidarestat etc.), nerve growth promoting agents (coleneuramide etc.) and the like.

### (3) Antihyperlipidemic Agents

Statin compounds which are cholesterol synthesis inhibitors (pravastatin, simvastatin, lovastatin, atorvastatin, fluvastatin, cerivastatin or their salts (sodium salt etc.) etc.), squalene synthase inhibitors or fibrate compounds having triglyceride lowering action (bezafibrate, clofibrate, simfibrate, clinofibrate etc.) and the like.

### (4) Hypotensive Agents

Angiotensin converting enzyme inhibitors (captopril, enalapril, delapril etc.), angiotensin II antagonists (losartan, candesartan cilexetil etc.), calcium antagonists (manidipine, nifedipine, amlodipine, efonidipine, nicardipine etc.), clonidine and the like.

### (5) Antiobesity Agents

Antiobesity drugs acting on the central nervous system (dexfenfluramine, fenfluramine, phentermine, sibutramine, anfepramon, dexamphetamine, mazindol, phenylpropanolamine, clobenzorex etc.), pancreatic lipase inhibitors (orlistat etc.), β3 agonists, anorectic peptides (leptin, CNTF (ciliary neurotrophic factor) etc.) and the like.

### (6) Therapeutic Drugs for Gastrointestinal Diseases

Anti-constipation drugs (lubiprostone etc.), gastric acid secretion inhibitors (H₂ blockers, proton pump inhibitors etc.), gastrointestinal motility improving drugs (alosetron, ramosetron, mosapride, tegaserod etc.).

### (7) Diuretic Agents

Xanthine derivatives (theobromine and sodium salicylate, theobromine and calcium salicylate etc.), thiazide preparations (ethiazide, cyclopenthiazide, trichlormethiazide, hydrochlorothiazide, hydroflumethiazide, benzylhydrochlorothiazide, penflutizide, polythiazide, methyclothiazide etc.), antialdosterone preparations (spironolactone, triamterene etc.), carbonate dehydratase inhibitors (acetazolamide etc.), chlorobenzenesulfonamide preparations (chlorthalidone, mefruside, indapamide etc.), azosemide, isosorbide, ethacrynic acid, piretanide, bumetanide, furosemide and the like.

### (8) Chemotherapeutic Agents

Alkylating agents (cyclophosphamide, ifosfamide etc.), metabolic antagonists (methotrexate, 5-fluorouracil etc.), carcinostatic antibiotics (mitomycin, adriamycin etc.), plant-derived carcinostatics (vincristine, vindesine, Taxol etc.), cisplatin, carboplatin, etoposide and the like. Particularly, 5-fluorouracil derivatives such as Furtulon, Neo-Furtulon and the like.

### (9) Immunotherapeutic Agents

Microorganism- or bacterium-derived components (muramyl dipeptide derivatives, Picibanil etc.), immunopotentiator polysaccharides (lentinan, schizophyllan, krestin etc.), genetically engineered cytokines (interferons, interleukins (IL) etc.), colony stimulating agents (granulocyte colony stimulating factor, erythropoietin etc.) and the like. Particularly, IL-1, IL-2, IL-12 and the like.

### (10) Pharmaceuticals Confirmed to Show Cachexia Improving Effect in Animal Model or Clinical Use

Progesterone derivatives (megestrol acetate etc.), metoclopramide pharmaceuticals, tetrahydrocannabinol pharmaceuticals (see, Journal of Clinical Oncology, vol. 12, pp. 213-225, 1994 as regards the aforementioned three pharmaceuticals), fat metabolism ameliorating agent (eicosapentaenoic acid etc.) (see British Journal of Cancer, vol. 68, pp. 314-318, 1993), growth hormone, IGF-1, antibodies against TNF-α, LIF, IL-6 and oncostatin M, which are the factors inducing cachexia, and the like.

### (11) Antiphlogistics

Steroidal agents (dexamethasone etc.), sodium hyaluronate, cyclooxygenase inhibitors (indomethacin, ketoprofen, loxoprofen, meloxicam, ampiroxicam, celecoxib, rofecoxib etc.) and the like.

### (12) Others

Glycosylation inhibitors, drugs acting on the central nervous system (antidepressants such as desipramine, amitriptyline, imipramine, fluoxetine, paroxetine, doxepin, carbamazepine etc.), anticonvulsants (lamotrigine etc.), antiarrhythmics (mexiletine etc.), endothelin receptor antagonists (atrasentan etc.), monoamine uptake inhibitors (tramadol etc.), indoleamine uptake inhibitors (e.g., fluoxetine, paroxetine), narcotic analgesics (morphine etc.), GABA receptor agonists (gabapentin etc.), GABA uptake inhibitors (tiagabine etc.), α₂ receptor agonists (clonidine etc.), topical analgesics (capsaicin etc.), protein kinase C inhibitors (ruboxistaurin etc.), antianxiety drugs (benzodiazepines etc.), antidepressants (amitriptyline, imipramine, clomipramine, dosulepin, amoxapine etc.), phosphodiesterase inhibitors (sildenafil etc.), dopamine receptor agonists (apomorphine etc.), anticholinergic agents, α₁ receptor blockers (tamsulosin etc.), muscle relaxants (baclofen etc.), potassium channel openers (nicorandil etc.), calcium channel blockers (nifedipine etc.), prophylactic and/or therapeutic drugs for Alzheimer's disease (donepezil, rivastigmine, galanthamine etc.), therapeutic drugs for Parkinson's disease (L-DOPA etc.), antithrombotics (aspirin, cilostazol etc.), NK2 receptor antagonists, therapeutic drugs for HIV infection (saquinavir, zidovudine, lamivudine, nevirapine etc.), therapeutic drugs for chronic obstructive pulmonary disease (salmeterol, tiotropium bromide, cilomilast etc.) and the like.

For the combined use of the compound of the present invention and the concomitant drug, the timing of the administration of the compound of the present invention and the concomitant drug is not restricted. The compound of the present invention and the concomitant drug can be administered to an administration subject simultaneously, or may be administered at different times. When administered in a staggered manner, the time difference varies depending on the active ingredient to be administered, dosage form and administration method. For example, when the concomitant drug is administered first, the compound of the present invention may be administered in 1 minute to 3 days, preferably 10 minutes to 1 day, more preferably 15 minutes to 1 hour, after the administration of the concomitant drug. When the compound of the present invention is administered first, the concomitant drug may be administered in 1 minute to 1 day, preferably 10 minutes to 6 hours, more preferably 15 minutes to 1 hour, after the administration of the compound of the present invention.

The administration mode of the compound of the present invention and the concomitant drug is not particularly restricted, and it is sufficient if the compound of the present invention and the concomitant drug are combined on administration. Examples of such administration mode include the following:

(1) The compound of the present invention and the concomitant drug are simultaneously produced to give a single preparation which is administered.
(2) The compound of the present invention and the concomitant drug are separately produced to give two kinds of preparations which are administered simultaneously by the same administration route.
(3) The compound of the present invention and the concomitant drug are separately produced to give two kinds of preparations which are administered by the same administration route only at the different times.
(4) The compound of the present invention and the concomitant drug are separately produced to give two kinds of preparations which are administered simultaneously by the different administration routes.
(5) The compound of the present invention and the concomitant drug are separately produced to give two kinds of preparations which are administered by the different administration routes only at different times (e.g., the compound of the present invention and the concomitant drug are administered in this order, or in the reverse order) and the like. In the following, these administration modes and concomitant agents themselves are collectively abbreviated as the concomitant agent of the present invention.

The concomitant agent of the present invention has low toxicity, and for example, the compound of the present invention or (and) the above-mentioned concomitant drug can be mixed, according to a method known per se, with a pharmacologically acceptable carrier to give pharmaceutical compositions, for example, tablets (including a sugar-coated tablet, film-coated tablet), powders, granules, capsules (including a soft capsule), solutions, injections, suppositories, sustained release agents and the like which can be safely administered orally or parenterally (local, rectum, vein, and the like).

As a pharmacologically acceptable carrier which may be used for preparing the concomitant agent of the present invention, those similar to the ones for the aforementioned pharmaceutical compositions of the present invention can be used.

The compounding ratio of the compound of the present invention to the concomitant drug in the concomitant agent of the present invention can be appropriately selected depending on the administration subject, administration route, diseases and the like.

The content of the compound of the present invention in the concomitant agent of the present invention differs depending on the form of a preparation, and usually in the range from about 0.01 to 99.99% by weight, preferably from about 0.1 to 50% by weight, further preferably from about 0.5 to 20% by weight, based on the whole preparation.

The content of the concomitant drug in the concomitant agent of the present invention differs depending on the form of the preparation, and is usually in the range from about 0.01 to 99.99% by weight, preferably from about 0.1 to 50% by weight, further preferably from about 0.5 to 20% by weight, based on the whole preparation.

The content of additives such as a carrier and the like in the concomitant agent of the present invention differs depending on the form of a preparation, and usually in the range from about 1 to 99.99% by weight, preferably from about 10 to 90% by weight, based on the whole preparation.

In the case when the compound of the present invention and the concomitant drug are separately prepared respectively, the same contents may be adopted.

The dose of the concomitant agent of the present invention varies depending on the kind of the compound of the present invention, age, body weight, condition, drug form, administration method, administration route, disease, administration period and the like, and can be appropriately selected. For example, for one patient suffering from irritable bowel syndrome (adult, body weight: about 60 kg), the combination agent is administered orally, generally at a dose of about 0.01 to 2000 mg/kg/day, preferably about 0.01 to 500 mg/kg/day, more preferably about 0.1 to about 100 mg/kg/day, particularly about 0.1 to about 50 mg/kg/day, especially about 1.5 to about 30 mg/kg/day, in terms of the compound of the present invention or the concomitant drug, respectively, once or divided several times in a day. Of course, since the dose as described above varies depending on various conditions, amounts smaller than the above-mentioned dose may sometimes be sufficient, further, amounts over that range sometimes have to be administered.

The amount of the concomitant drug can be set at any value unless its side effects are problematical. The daily dose in terms of the concomitant drug differs depending on the severity, age, sex, body weight, sensitivity difference of the subject, administration period, interval, and nature, pharmacology, kind of the pharmaceutical preparation, kind of effective ingredient, and the like, and not particularly restricted, and the amount of the drug is, in the case of oral administration, for example, usually in the range from about 0.001 to 2000 mg, preferably from about 0.01 to 500 mg, further preferably from about 0.1 to 100 mg, per 1 kg body weight of a mammal and this is usually administered once to 4 portions divided for one day.

### EXAMPLES

The present invention is hereinafter explained in more detail by means of the following Reference Examples, Examples, Preparation Examples and Experimental Examples, which are not to be construed as limitative.

The compounds to be the starting materials can be synthesized and used according to the methods described in WO2008/153027 and WO2005/105802, or methods analogous thereto.

In the following Reference Examples and Examples, “room temperature” generally means the range of from about 10° C. to about 35° C. “%” means weight percent, unless otherwise specified. LCMS (ESI or APCI) was measured using Waters LC-MS systems (ZQ, ZMD-1, ZMD-2) or Agilent G6100 series LC/MSD system. Hydrogen nuclear magnetic resonance (¹H-NMR) spectrum was measured using Varian Mercury-300 hydrogen nuclear magnetic resonance apparatus (300 MHz) or Bruker Ultra Shield-300 hydrogen nuclear magnetic resonance apparatus (300 MHz), and shown in δ value (ppm) using tetramethylsilane (TMS) as an internal standard. Elemental analysis of chlorine or bromine was performed using ICS-1500 (manufactured by Dionex), and elemental analysis of carbon, hydrogen and nitrogen was performed using Vario EL (manufactured by elementar) or Vario MICRO CUBE (manufactured by elementar). Water content was measured (coulometric titration method) using AQ-2100 (HIRANUMA) Karl Fischer coulometric titrator.

### Reference Example 1

### Synthesis of ethyl (1S,2R)-5,5-difluoro-2-({[4-(3-methyl-1H-pyrazol-1-yl)phenyl]carbonyl}amino)cyclohexanecarboxylate

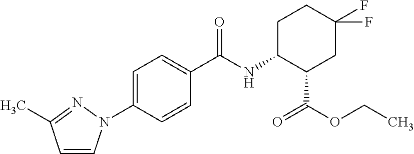

A mixture of ethyl (1S,2R)-2-amino-5,5-difluorocyclohexanecarboxylate (7.35 g), 4-(3-methyl-1H-pyrazol-1-yl)benzoic acid (7.89 g), triethylamine (9.89 g), N-[3-(dimethylamino)propyl]-N′-ethylcarbodiimide hydrochloride (7.48 g), 1H-1,2,3-benzotriazol-1-ol (2.72 g) and acetonitrile (355 mL) was stirred at room temperature for 12 hr. To the reaction mixture was added saturated aqueous sodium hydrogen carbonate solution, and the mixture was extracted with ethyl acetate, washed with brine, and dried over anhydrous magnesium sulfate. The desiccant was filtered off, and the filtrate was concentrated under reduced pressure. The residue was purified by recrystallization (recrystallization solvent: ethyl acetate, hexane) to give the title compound (13.0 g) as colorless crystals.

¹H-NMR (DMSO-d₆) δ: 1.08 (3H, t, J=7.0 Hz), 1.76-2.02 (3H, m), 2.03-2.33 (5H, m), 2.61 (1H, dd, J=33.5, 3.2 Hz), 2.89-3.05 (1H, m), 3.85-4.19 (2H, m), 4.67-4.81 (1H, m), 6.38 (1H, d, J=2.3 Hz), 7.77-7.95 (4H, m), 8.27 (1H, d, J=9.1 Hz), 8.47 (1H, d, J=2.7 Hz).

### Reference Example 2

### Synthesis of (1S,2R)-5,5-difluoro-2-({[4-(3-methyl-1H-pyrazol-1-yl)phenyl]carbonyl}amino)cyclohexanecarboxylic acid

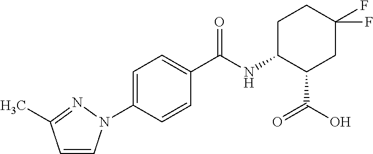

To a solution of ethyl (1S,2R)-5,5-difluoro-2-({[4-(3-methyl-1H-pyrazol-1-yl)phenyl]carbonyl}amino)cyclohexanecarboxylate (13.3 g) in methanol (170 mL) was added 1N aqueous sodium hydroxide solution (70 mL) and the mixture was stirred at room temperature for 12 hr. Methanol was evaporated under reduced pressure, and the residue was washed with ether, and neutralized with 1N hydrochloric acid. The organic layer was extracted with ethyl acetate, washed with brine, and dried over anhydrous magnesium sulfate. The desiccant was filtered off and the filtrate was concentrated under reduced pressure.

The residue was purified by recrystallization (recrystallization solvent: diisopropyl ether) to give the title compound (9.00 g) as colorless crystals.

¹H-NMR (DMSO-d₆) δ: 1.73-2.24 (5H, m), 2.28 (3H, s), 2.53-2.84 (1H, m), 2.85-2.97 (1H, m), 4.68-4.78 (1H, m), 6.38 (1H, d, J=2.3 Hz), 7.81-8.01 (4H, m), 8.23 (1H, d, J=9.1 Hz), 8.47 (1H, d, J=2.7 Hz), 12.45 (1H, s).

### Reference Example 3

### Synthesis of {2-{(3aR*,4R*,9bR*)-1-{[(1S,2R)-5,5-difluoro-2-({[4-(3-methyl-1H-pyrazol-1-yl)phenyl]carbonyl}amino)cyclohexyl]carbonyl}-2,3,3a,4,5,9b-hexahydro-1H-pyrrolo[3,2-c]quinolin-4-yl]-1H-imidazol-1-yl}methyl 2,2-dimethylpropanoate

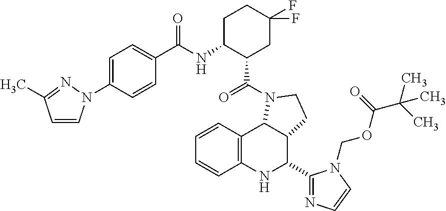

{2-[(3aS*,4R*,9bR*)-2,3,3a,4,5,9b-Hexahydro-1H-pyrrolo[3,2-c]quinolin-4-yl]-1H-imidazol-1-yl}methyl 2,2-dimethylpropanoate dihydrochloride (1.00 g), (1S,2R)-5,5-difluoro-2-({[4-(3-methyl-1H-pyrazol-1-yl)phenyl]carbonyl}amino)cyclohexanecarboxylic acid (0.850 g) and triethylamine (0.978 mL) were mixed in N,N-dimethylformamide (20 mL). The reaction mixture was cooled to 0° C., diethyl cyanophosphate (0.434 mL) was added, and the mixture was stirred at room temperature for 12 hr. Water was added to the reaction mixture, and the mixture was extracted with ethyl acetate, washed with brine, and dried over anhydrous magnesium sulfate. The desiccant was filtered off and the filtrate was concentrated under reduced pressure. The residue was purified by silica gel column chromatography (eluent ethyl acetate:hexane=50:50-100:0) to give the title compound (1.38 g) as an amorphous solid.

LCMS, m/z 700(M+1)

### Reference Example 4

### Synthesis of N-[(1R,2S)-4,4-difluoro-2-{[(3aR*,4R*,9bR*)-4-(1H-imidazol-2-yl)-2,3,3a,4,5,9b-hexahydro-1H-pyrrolo[3,2-c]quinolin-1-yl]carbonyl}cyclohexyl]-4-(3-methyl-1H-pyrazol-1-yl)benzamide

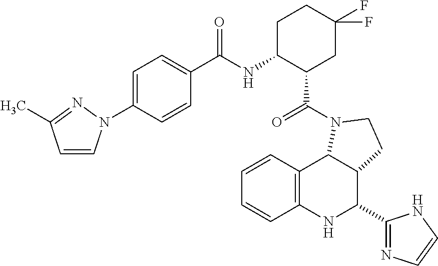

A mixture of {2-[(3aR*,4R*,9bR*)-1-{[(1S,2R)-5,5-difluoro-2-({[4-(3-methyl-1H-pyrazol-1-yl)phenyl]carbonyl}amino)cyclohexyl]carbonyl}-2,3,3a,4,5,9b-hexahydro-1H-pyrrolo[3,2-c]quinolin-4-yl]-1H-imidazol-1-yl}methyl 2,2-dimethylpropanoate (1.38 g), 28% aqueous ammonia (10 mL) and methanol (10 mL) was stirred at room temperature for 12 hr. The reaction mixture was concentrated under reduced pressure, and the residue was dissolved in ethyl acetate, washed with brine, and dried over anhydrous magnesium sulfate. The desiccant was filtered off and the filtrate was concentrated under reduced pressure. The residue was purified by silica gel column chromatography (eluent ethyl acetate:hexane=50:50-100:0) to give the title compound (680 mg) as an amorphous solid.

LCMS, m/z 586(M+1)

### Reference Example 5

### Synthesis of tert-butyl (3aR*,4R*,9bR*)-4-(1-{[(2,2-dimethylpropanoyl)oxy]methyl}-1H-imidazol-2-yl)-8-fluoro-2,3,3a,4,5,9b-hexahydro-1H-pyrrolo[3,2-c]quinoline-1-carboxylate

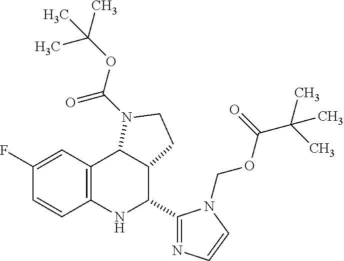

A mixture of (2-formyl-1H-imidazol-1-yl)methyl 2,2-dimethylpropanoate (1.9 g), 4-fluoroaniline (1 g) and toluene (30 mL) was stirred under ice-cooling for 2 hr. To the reaction mixture were added tert-butyl 2,3-dihydro-1H-pyrrole-1-carboxylate (2.5 g) and dysprosium tris(trifluoromethanesulfonate) (Dy(OTf)₃) (0.27 g), and the mixture was stirred at room temperature for 10 hr. The reaction mixture was concentrated under reduced pressure, and water was added. The mixture was extracted with ethyl acetate, washed with brine, and dried over anhydrous magnesium sulfate. The desiccant was filtered off and the filtrate was concentrated under reduced pressure. The residue was purified by silica gel column chromatography (eluent ethyl acetate:hexane=3:7) to give the title compound (411 mg) as an amorphous solid.

¹H-NMR (CDCl₃) δ: 1.17-1.22 (9H, m), 1.48-1.62 (9H, m), 1.70-1.85 (1H, m), 2.07-2.25 (1H, m), 2.60-2.78 (1H, m), 3.16-3.47 (2H, m), 4.30-4.55 (1H, m), 4.80-5.00 (1H, m), 5.25-5.40 (1H, m), 5.79-6.05 (2H, m), 6.51-7.50 (5H, m).

### Reference Example 6

### Synthesis of {2-[(3aS*,4R*,9bR*)-8-fluoro-2,3,3a,4,5,9b-hexahydro-1H-pyrrolo[3,2-c]quinolin-4-yl]-1H-imidazol-1-yl}methyl 2,2-dimethylpropanoate dihydrochloride

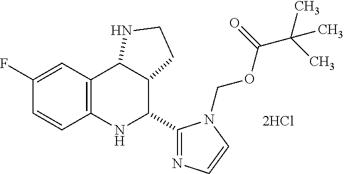

To a solution of tert-butyl (3aR*,4R*,9bR*)-4-(1-1-{[(2,2-dimethylpropanoyl)oxy]methyl}-1H-imidazol-2-yl)-8-fluoro-2,3,3a,4,5,9b-hexahydro-1H-pyrrolo[3,2-c]quinoline-1-carboxylate (411 mg) in methanol (10 mL) was added a solution of 4 N hydrogen chloride in ethyl acetate (1.74 mL) at 0° C., and the mixture was stirred at room temperature for 12 hr, and concentrated under reduced pressure to give the title compound (321 mg) as an amorphous solid.

LCMS, m/z 373(M+1)

¹H-NMR (DMSO-d₆) δ: 0.94-1.27 (10H, m), 1.56-1.76 (1H, m), 1.99 (2H, s), 2.92-3.23 (3H, m), 5.16 (2H, dd, J=58.6, 3.2 Hz), 6.07-6.33 (1H, m), 6.82 (1H, dd, J=9.0, 5.3 Hz), 7.00-7.12 (1H, m), 7.22-7.53 (1H, m), 7.82 (1H, s).

### Reference Example 7

### Synthesis of {2-[(3aR*,4R*,9bR*)-1-{[(1S,2R)-5,5-difluoro-2-({[4-(3-methyl-1H-pyrazol-1-yl)phenyl]carbonyl}amino)cyclohexyl}carbonyl}-8-fluoro-2,3,3a,4,5,9b-hexahydro-1H-pyrrolo[3,2-c]quinolin-4-yl]-1H-imidazol-1-yl}methyl 2,2-dimethylpropanoate

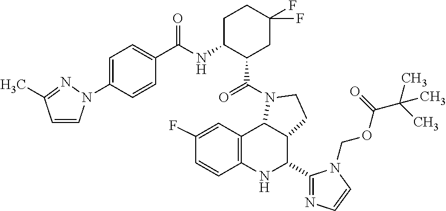

Using {2-[(3aS*,4R*,9bR*)-8-fluoro-2,3,3a,4,5,9b-hexahydro-1H-pyrrolo[3,2-c]quinolin-4-yl]-1H-imidazol-1-yl}methyl 2,2-dimethylpropanoate dihydrochloride and (1S,2R)-5,5-difluoro-2-({[4-(3-methyl-1H-pyrazol-1-yl)phenyl]carbonyl}amino)cyclohexanecarboxylic acid and in the same manner as in Reference Example 3, the title compound was synthesized.

LCMS, m/z 718(M+1)

### Reference Example 8

### Synthesis of N-[(1R,2S)-4,4-difluoro-2-{[(3aR*,4R*,9bR*)-8-fluoro-4-(1H-imidazol-2-yl)-2,3,3a,4,5,9b-hexahydro-1H-pyrrolo[3,2-c]quinolin-1-yl]carbonyl}cyclohexyl]-4-(3-methyl-1H-pyrazol-1-yl)benzamide

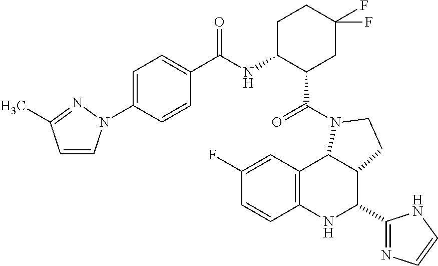

Using {2-[(3aR*, 4R*,9bR*)-1-{[(1S,2R)-5,5-difluoro-2-({[4-(3-methyl-1H-pyrazol-1-yl)phenyl]carbonyl}amino)cyclohexyl]carbonyl)-8-fluoro-2,3,3a,4,5,9b-hexahydro-1H-pyrrolo[3,2-c]quinolin-4-yl]-1H-imidazol-1-yl}methyl 2,2-dimethylpropanoate and in the same manner as in Reference Example 4, the title compound was synthesized.

LCMS, m/z 604(M+1)

### Reference Example 9

### Synthesis of (3aS*,4R*,9bR*)-4-thiophen-3-yl-2,3,3a,4,5,9b-hexahydro-1H-pyrrolo[3,2-c]quinoline dihydrochloride

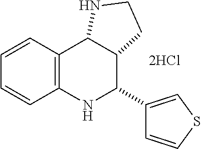

Using tert-butyl (3aR*,4R*,9bR*)-4-thiophen-3-yl-2,3,3a,4,5,9b-hexahydro-1H-pyrrolo[3,2-c]quinoline-1-carboxylate and in the same manner as in Reference Example 6, the title compound was synthesized.

LCMS, m/z 257(M+1)

### Reference Example 10

### Synthesis of N-[(1R,2S)-4,4-difluoro-2-{[(3aR*,4R*,9bR*)-4-thiophen-3-yl-2,3,3a,4,5,9b-hexahydro-1H-pyrrolo[3,2-c]quinolin-1-yl]carbonyl}cyclohexyl]-4-(3-methyl-1H-pyrazol-1-yl)benzamide

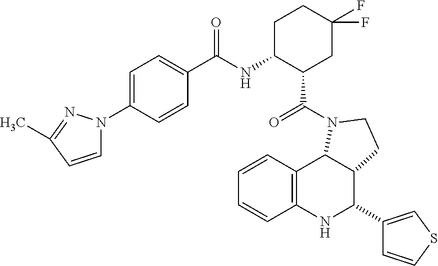

Using (3aS*,4R′,9bR*)-4-thiophen-3-yl-2,3,3a,4,5,9b-hexahydro-1H-pyrrolo[3,2-c]quinoline dihydrochloride and (1S,2R)-5,5-difluoro-2-({[4-(3-methyl-1H-pyrazol-1-yl)phenyl]carbonyl}amino)cyclohexanecarboxylic acid and in the same manner as in Reference Example 3, the title compound was synthesized.

¹H-NMR (CDCl₃) δ: 1.58-2.27 (7H, m), 2.33-2.70 (5H, m), 2.75-2.90 (1H, m), 3.05-6.65 (6H, m), 6.70-7.50 (8H, m), 7.70-7.99 (6H, m).

### Reference Example 11

### Synthesis of N-[(1R,2S)-4,4-difluoro-2-{[(3aR*,4R*,9bR*)-8-fluoro-4-phenyl-2,3,3a,4,5,9b-hexahydro-1H-pyrrolo[3,2-c]quinolin-1-yl]carbonyl}cyclohexyl]-4-(3-methyl-1H-pyrazol-1-yl)benzamide

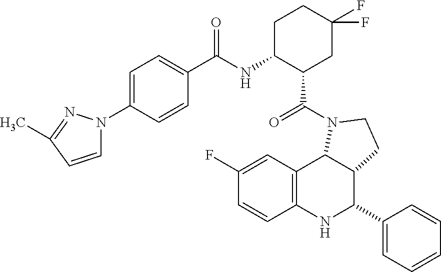

Using (3aS*,4R*,9bR*)-8-fluoro-4-phenyl-2,3,3a,4,5,9b-hexahydro-1H-pyrrolo[3,2-c]quinoline dihydrochloride and (1S,2R)-5,5-difluoro-2-({[4-(3-methyl-1H-pyrazol-1-yl)phenyl]carbonyl}amino)cyclohexanecarboxylic acid and in the same manner as in Reference Example 3, the title compound was synthesized.

LCMS, m/z 614(M+1)

### Reference Example 12

### Synthesis of (2-formyl-1H-imidazol-1-yl)methyl 2,2-dimethylpropanoate

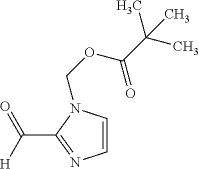

To a mixture of 1H-imidazole-2-carbaldehyde (150 g), potassium carbonate (258 g) and N,N-dimethylformamide (3 L) was added chloromethyl 2,2-dimethylpropanoate (259 g) at room temperature, and the mixture was stirred at 55° C. for 18 hr. The mixture was cooled to room temperature, the insoluble material was filtered off, and the filtrate was concentrated. The residue was diluted with ethyl acetate (2.25 L), washed with 5% aqueous sodium hydrogen carbonate solution (1.5 L) and brine (0.75 L), dried over anhydrous sodium sulfate and concentrated. The residue was purified by silica gel column chromatography (elution solvent ethyl acetate:hexane=30:70-40:60) to give the title compound (291 g) as an oil.

¹H-NMR (CDCl₃) δ: 1.17 (9H, s), 6.30 (2H, s), 7.28-7.31 (1H, m), 7.36-7.40 (1H, m), 9.84-9.86 (1H, m).

### Reference Example 13

### Synthesis of tert-butyl (3aR*,4R*,9bR*)-4-(1-{[(2,2-dimethylpropanoyl)oxy]methyl}-1H-imidazol-2-yl)-2,3,3a,4,5,9b-hexahydro-1H-pyrrolo[3,2-c]quinoline-1-carboxylate

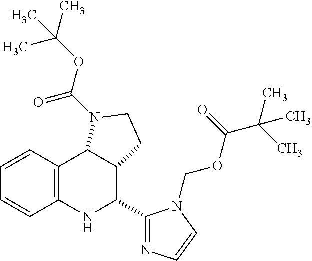

To a solution of aniline (28.6 g) in toluene (1050 mL) were added anhydrous magnesium sulfate (111 g) and (2-formyl-1H-imidazol-1-yl)methyl 2,2-dimethylpropanoate (70 g), and the mixture was stirred at room temperature for 3 hr. To the reaction mixture was added dysprosium tris(trifluoromethanesulfonate) (37.4 g), and a solution of tert-butyl 2,3-dihydro-1H-pyrrole-1-carboxylate (69 g) in toluene (350 mL) was added dropwise over 1 hr. After stirring for 12 hr, the reaction mixture was filtered, and the filtrate was directly purified by silica gel column chromatography (2.1 kg, elution solvent ethyl acetate:hexane=1:3-3:2) to give the title compound (51.7 g) as a pale-brown amorphous solid.

¹H-NMR (CDCl₃) δ: 1.19 (9H, s), 1.46-1.63 (9H, m), 1.66-1.85 (1H, m), 2.13-2.34 (1H, m), 2.62-2.83 (1H, m), 3.20-3.57 (2H, m), 4.46-4.63 (1H, m), 4.92-5.04 (1H, m), 5.25-5.49 (1H, m), 5.77-5.93 (1H, m), 5.96-6.07 (1H, m), 6.57-6.67 (1H, m), 6.71-6.82 (1H, m), 6.98-7.16 (3H, m), 7.45-7.74 (1H, m).

### Reference Example 14

### Synthesis of (2-[(3aS*,4R*,9bR*)-2,3,3a,4,5,9b-hexahydro-1H-pyrrolo[3,2-c]quinolin-4-yl]-1H-imidazol-1-yl}methyl 2,2-dimethylpropanoate dihydrochloride

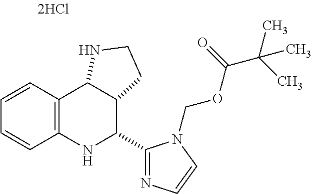

tert-Butyl (3aR*,4R*,9bR*)-4-({[(2,2-dimethylpropanoyl)oxy]methyl}-1H-imidazol-2-yl)-2,3,3a,4,5,9b-hexahydro-1H-pyrrolo[3,2-c]quinoline-1-carboxylate obtained in Reference Example 13 was diluted with ethyl acetate, and the solution was washed with brine, dried over sodium sulfate, and concentrated. The residue (92 g) was dissolved in acetonitrile (460 mL), and under ice-cooling, a solution of 2N hydrogen chloride in ethyl acetate (920 mL) was slowly added dropwise. After stirring at room temperature for 3.5 hr, ethyl acetate (180 mL) was added dropwise to the reaction mixture, and the mixture was further stirred for 18 hr. Ethyl acetate (460 mL) was further added dropwise to this mixture, and the mixture was further stirred for 3 hr, and then stirred for 2 hr under ice-cooling. Under a nitrogen stream, the precipitate was collected by filtration, and washed with acetonitrile/ethyl acetate mixed solvent (1:3.4, 3.7 L) to give crude crystals. A similar reaction operation was performed again using tert-butyl (3aR*,4R*,9bR*)-4-(1-{[(2,2-dimethylpropanoyl)oxy]methyl}-1H-imidazol-2-yl)-2,3,3a,4,5,9b-hexahydro-1H-pyrrolo[3,2-c]quinoline-1-carboxylate (92 g), and the 2 batches were combined to give crude crystals (156 g). The obtained crude crystals (78 g) were dissolved in methanol (234 mL) at 55° C. and, at the same temperature, ethyl acetate (650 mL) was slowly added dropwise and the mixture was stirred for 1 hr. Ethyl acetate (520 mL) was further added dropwise at 55° C., and the mixture was stirred at the same temperature for 1 hr and at room temperature for 18 hr. The precipitate was collected by filtration, washed with methanol/ethyl acetate mixed solvent (10:90), and dried to give the title compound. The remaining crude crystals (78 g) were recrystallized by a similar operation, and the 2 batches were combined to give 138 g of the title compound.

¹H-NMR (DMSO-d₆) δ: 1.10-1.21 (9H, m), 1.73 (1H, brs), 1.87-2.10 (1H, m), 2.93-3.24 (3H, m), 5.01 (1H, brs), 5.24 (1H, brs), 6.09-6.19 (1H, m), 6.21-6.33 (1H, m), 6.58-6.73 (1H, m), 6.74-6.91 (2H, m), 7.11-7.23 (1H, m), 7.43 (1H, d, J=7.3 Hz), 7.57 (1H, brs), 7.72-7.89 (1H, m), 10.80 (1H, brs).

### Reference Example 15

### Synthesis of ethyl 8-{[(1R)-1-phenylethyl]amino)-1,4-dioxaspiro[4.5]dec-7-ene-7-carboxylate

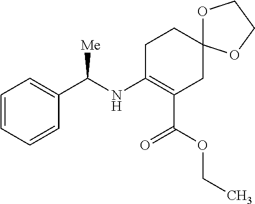

A mixture of ethyl 8-hydroxy-1,4-dioxaspiro[4.5]dec-7-ene-7-carboxylate (230 g), (1R)-1-phenylethanamine (147 g) and p-toluenesulfonic acid monohydrate (9.58 g) in toluene (1.6 L) was stirred at 70° C. for 1 hr, and heated under reflux for 16 hr while dehydrating with a Dean-Stark trap. The reaction mixture was concentrated, and the residue was purified by silica gel column chromatography (elution solvent hexane:ethyl acetate=10:1-6:1) to give the title compound (290 g) as a yellow solid.

¹H-NMR (CDCl₃) δ: 1.28 (3H, t, J=7.0 Hz), 1.48 (3H, d, J=6.8 Hz), 1.58-1.73 (2H, m), 2.21 (1H, dt, J=14.4, 6.3 Hz), 2.46-2.59 (3H, m), 3.87-4.03 (4H, m), 4.14 (2H, q, J=6.9 Hz), 4.58-4.65 (1H, m), 7.21-7.35 (5H, m), 9.41 (1H, d, J=7.2 Hz).

### Reference Example 16

### Synthesis of ethyl (7S,8R)-8-{[(1R)-1-phenylethyl]amino}-1,4-dioxaspiro[4.5]decane-7-Carboxylate hydrobromide

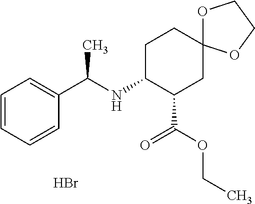

Sodium borohydride (73.2 g) was added in small portions to isobutyric acid (1220 mL) at 0° C. The reaction mixture was stirred at room temperature for 30 min, cooled to 0° C., and a solution of ethyl 8-{[(1R)-1-phenylethyl]amino}-1,4-dioxaspiro[4.5]dec-7-ene-7-carboxylate (200 g) in isobutyric acid (300 mL) was added dropwise at the same temperature. The mixture was stirred at room temperature for 18 hr, ice-cooled to 0-10° C., and basified (pH=9) with water (400 mL) and 8N aqueous sodium hydroxide solution (1.75 L). The organic layer was extracted with ethyl acetate (2.5 L), water (2.5 L) was added to the aqueous layer and the mixture was extracted again with ethyl acetate (2.5 L). The combined organic layer was washed with 1N aqueous sodium hydroxide solution (1.6 L and 0.8 L) and brine (1.6 L), and dried over magnesium sulfate. After filtration, the filtrate was subjected to NH-silica gel column chromatography (1.0 kg, elution solvent ethyl acetate) and concentrated. The obtained residue was dissolved in isopropyl acetate (2.1 L), ice-cooled to 0-10° C., and a solution of 25% hydrogen bromide in acetic acid (199 g) diluted with isopropyl acetate (200 mL) was added dropwise. The mixture was stirred for 2 hr and the resulting crystals were collected by filtration, washed with isopropyl acetate (500 mL) and dried to give crude crystals (208 g). The thus-obtained crude crystals (293 g) were dissolved in ethanol (310 mL) and ethyl acetate (310 mL) at 70° C. To this solution was slowly added dropwise ethyl acetate (735 mL) at the same temperature, and then heptane (735 mL) was slowly added dropwise to allow crystal precipitation. The suspension was stirred at the same temperature for 30 min and at room temperature for 1 hr. The obtained crystals were collected by filtration, washed with ethyl acetate/hexane mixed solvent (1:1, 200 mL), and dried to give 230 g of crude crystals. The obtained crude crystals (229 g) were dissolved in ethanol (273 mL) and ethyl acetate (273 mL) at 70° C. To this solution was slowly added dropwise ethyl acetate (572 mL) at the same temperature, and then heptane (572 mL) was slowly added dropwise to allow crystal precipitation. The suspension was stirred at 70° C. for 30 min, and heptane (572 mL) was again slowly added dropwise. The mixture was stirred at 70° C. for 30 min, at room temperature for 1 hr, and at 0° C. for 1 hr. The obtained crystals were collected by filtration, washed with ethyl acetate/hexane mixed solvent (1:1, 500 mL), and dried to give 185 g of crude crystals. The thus-obtained crude crystals (250 g) were dissolved in ethanol (250 mL) and ethyl acetate (250 mL) at 70° C. To this solution was slowly added dropwise heptane (750 mL) at the same temperature to allow crystal precipitation. After stirring at the same temperature for 30 min, to this suspension was added dropwise heptane (750 mL), and the mixture was further stirred for 30 min and then at room temperature for 2 hr. The obtained crystals were collected by filtration, washed with ethyl acetate/hexane mixed solvent (1:2, 500 mL), and dried to give the title compound (227 g) as colorless crystals.

¹H-NMR (DMSO-d₆) δ: 1.26 (3H, t, J=7.1 Hz), 1.30-1.79 (7H, m), 1.85-2.07 (1H, m), 2.13-2.28 (1H, m), 3.15-3.33 (2H, m), 3.66-3.80 (2H, m), 3.81-3.95 (2H, m), 4.16 (2H, m), 4.48 (1H, m), 7.36-7.52 (3H, m), 7.67 (2H, dd, J=7.6, 1.8 Hz), 8.58-9.10 (2H, m).

### Reference Example 17

### Synthesis of ethyl (7S,8R)-8-amino-1,4-dioxaspiro[4.5]decane-7-carboxylate hydrobromide

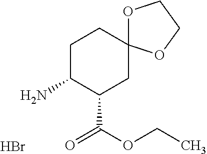

Ethyl (7S,8R)-8-{[(1R)-1-phenylethyl]amino}-1,4-dioxaspiro[4.5]decane-7-carboxylate hydrobromide (118 g) was dissolved in ethanol (1.2 L), 10% palladium carbon (50 wt % wet with water, 11.8 g) was added under a nitrogen atmosphere, and the mixture was stirred at 50° C. under a hydrogen atmosphere for 5 hr. The reaction system was purged with nitrogen gas, the reaction mixture was filtered, and the filtrate was concentrated under reduced pressure. A similar reaction operation was performed 3 times using ethyl (7S,8R)-8-{[(1R)-1-phenylethyl]amino-1-1,4-dioxaspiro[4.5]decane-7-carboxylate hydrobromide (118 g, 118 g and 118 g), and 4 batches in total were combined to give the title compound (353 g).

¹H-NMR (CDCl₃) δ: 1.30 (3H, t, J=7.2 Hz), 1.67-1.97 (3H, m), 2.18-2.34 (2H, m), 2.43 (1H, ddd, J=14.1, 5.0, 2.2 Hz), 3.35 (1H, q, J=4.8 Hz), 3.63-3.78 (1H, m), 3.80-4.04 (4H, m), 4.11-4.31 (2H, m).

### Reference Example 18

### Synthesis of ethyl (1S,2R)-2-{[(benzyloxy)carbonyl]amino}-5-oxocyclohexanecarboxylate

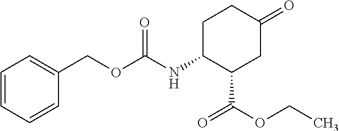

To ethyl (7S,8R)-8-amino-1,4-dioxaspiro[4.5]decane-7-carboxylate hydrobromide (353 g) were added tetrahydrofuran (THF) (1.8 L), sodium carbonate (362 g) and water (900 mL) under ice-cooling. Benzyl chlorocarbonate (215 g) was added at the same temperature, and the mixture was stirred at room temperature for 4 hr and extracted with ethyl acetate (2 L). The organic layer was washed with 1N hydrochloric acid (1.5 L), and further washed with brine (1.5 L). The organic layer was dried over magnesium sulfate, and filtered, and the filtrate was concentrated to give ethyl (7S,8R)-8-{[(benzyloxy)carbonyl]amino}-1,4-dioxaspiro[4.5]decane-7-carboxylate. The resultant product was divided into 2 batches, each was dissolved in acetone (1.75 L), and 4N hydrochloric acid (875 mL) was added under ice-cooling. The reaction mixture was stirred at room temperature for 18 hr, acetone was evaporated under reduced pressure, and the resultant aqueous layer was extracted with ethyl acetate (1 L). The extract was washed with brine (1 L) and concentrated. The residue was purified by silica gel column chromatography (1.0 kg, elution solvent ethyl acetate:hexane=1:3-1:1), and 2 batches were combined to give the title compound (353 g).

¹H-NMR (CDCl₃) δ: 1.23 (3H, t, J=7.1 Hz), 2.02-2.13 (3H, m), 2.28-2.58 (2H, m), 2.64-2.78 (1H, m), 3.10-3.23 (1H, m), 4.15 (2H, q, J=7.0 Hz), 4.24-4.43 (1H, m), 5.03-5.20 (2H, m), 5.63 (1H, d, J=8.3 Hz), 7.21-7.45 (5H, m).

### Reference Example 19

### Synthesis of ethyl (1S,2R)-2-{[(benzyloxy)carbonyl]amino}-5,5-difluorocyclohexanecarboxylate

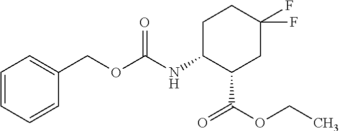

Ethyl (1S,2R)-2-{[(benzyloxy)carbonyl]amino}-5-oxocyclohexanecarboxylate (103 g) was dissolved in toluene (1.5 L), Deoxo-Fluor® (143 g) was slowly added dropwise under ice-cooling, and the mixture was stirred at room temperature for 3 hr. Under ice-cooling, water (500 mL) was slowly added dropwise to the reaction mixture, and an aqueous solution (1.0 L) of sodium carbonate (150 g) was slowly added dropwise. At the same temperature, powder calcium chloride (150 g) was slowly added, and the mixture was stirred at room temperature for 45 min. To the reaction mixture was added water (1 L), and the mixture was extracted with ethyl acetate (2.0 L), and further extracted with ethyl acetate (1.5 L). The combined organic layer was washed with brine (1.5 L), dried over magnesium sulfate, and concentrated to give a crude product. A similar reaction was performed using 115 g and 105 g of ethyl (1S,2R)-2-{[(benzyloxy)carbonyl]amino}-5-oxocyclohexanecarboxylate, and 3 bathes in total were combined to give a crude product (332 g). From the obtained crude product, 166 g was dissolved in acetonitrile (1.0 L) and water (1.0 L), ruthenium chloride (1.05 g) was added under ice-cooling, sodium periodate (108 g) was slowly added at the same temperature, and the mixture was stirred at room temperature for 18 hr. The organic layer of the reaction mixture was extracted with ethyl acetate (2 L), and further extracted with ethyl acetate (1.0 L). The combined extract was washed with saturated aqueous sodium thiosulfate solution (2.0 L), saturated aqueous sodium hydrogen carbonate solution (2.0 L) and brine (2.0 L), dried over magnesium sulfate, and concentrated under reduced pressure. The residue was purified by silica gel column chromatography (1.5 kg, elution solvent ethyl acetate:hexane=5:95-15:85) to give the title compound. A similar reaction operation was performed using the remaining crude product (166 g), and 2 batches were combined to give the title compound (163 g).

¹H-NMR (CDCl₃) δ: 1.18-1.31 (3H, m), 1.75-2.02 (3H, m), 2.06-2.25 (2H, m), 2.29-2.52 (1H, m), 2.92 (1H, dt, J=7.3, 4.8 Hz), 3.99-4.28 (3H, m), 5.09 (2H, s), 5.56 (1H, brs), 7.28-7.41 (5H, m).

### Reference Example 20

### Synthesis of 4-(3-methyl-1H-pyrazol-1-yl)benzoic acid

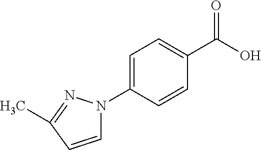

To a solution of 4-hydrazinobenzoic acid (75 g) in ethanol (1.5 L) was slowly added 4,4-dimethoxybutan-2-one (65 g) at room temperature. The reaction mixture was heated under reflux for 12 hr, and cooled to room temperature. The precipitated crystals were collected by filtration and dried. The obtained crystals were dissolved in methanol (540 mL) and THF (2.16 L), activated carbon (15 g) was added and the mixture was stirred at room temperature for 2 hr. The activated carbon was filtered off, and the filtrate was concentrated. The residue was recrystallized from ethanol (2.6 L) to give the title compound (61.3 g).

¹H-NMR (DMSO-d₆) δ: 2.30 (3H, s), 6.40 (1H, d, J=2.4 Hz), 7.89-7.97 (2H, m), 7.99-8.09 (2H, m), 8.50 (1H, d, J=2.6 Hz), 12.98 (1H, brs).

### Reference Example 21

### Synthesis of ethyl (1S,2R)-2-amino-5,5-difluorocyclohexanecarboxylate

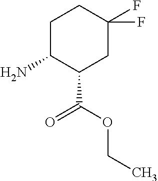

To a solution of ethyl (1S,2R)-2-{[(benzyloxy)carbonyl]amino}-5,5-difluorocyclohexanecarboxylate (68 g) in ethanol (0.7 L) was added 10% palladium carbon (50 wt % wet with water, 13.6 g), and the mixture was stirred at room temperature under a hydrogen atmosphere for 4 hr. The reaction mixture was filtered, and the filtrate was concentrated under reduced pressure. A similar reaction operation was performed using ethyl (1S,2R)-2-{[(benzyloxy)carbonyl]amino}-5,5-difluorocyclohexanecarboxylate (68 g), and 2 batches were combined to give the title compound (77 g).

¹H-NMR (CDCl₃) δ: 1.29 (3H, t, J=7.2 Hz), 1.74-2.49 (6H, m), 2.70-2.81 (1H, m), 3.61 (1H, brs), 4.19 (2H, q, J=7.0 Hz).

### Reference Example 22

### Synthesis of ethyl (1S,2R)-5,5-difluoro-2-({(4-(3-methyl-1H-pyrazol-1-yl)phenyl]carbonyl}amino)cyclohexanecarboxylate

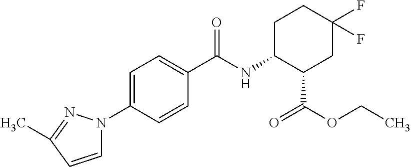

To a suspension of ethyl (1S,2R)-2-amino-5,5-difluorocyclohexanecarboxylate (77 g), 4-(3-methyl-1H-pyrazol-1-yl)benzoic acid (85 g) and acetonitrile (1 L) were added triethylamine (42.3 g), N-[3-(dimethylamino)propyl]-N′-ethylcarbodiimide hydrochloride (91.6 g) and 1H-1,2,3-benzotriazol-1-ol (30.5 g) under ice-cooling. After stirring at room temperature for 18 hr, water (1 L) and ethyl acetate (1 L) were added to the reaction mixture. The organic layer was extracted, and extracted again with ethyl acetate (1 L). The combined extract was washed with saturated aqueous sodium hydrogen carbonate solution (1 L) and brine (1 L), dried over magnesium sulfate, and filtered by NH-silica gel column chromatography (700 g, elution solvent ethyl acetate). The filtrate was concentrated, and the residue was washed with diisopropyl ether (1 L), and dried to give the title compound (134 g).

¹H-NMR (CDCl₃) δ: 1.29 (3H, t, J=7.2 Hz), 1.84-2.35 (5H, m), 2.39 (3H, s), 2.54-2.77 (1H, m), 3.04 (1H, q, J=5.0 Hz), 4.07-4.34 (2H, m), 4.41-4.56 (1H, m), 6.29 (1H, d, J=2.4 Hz), 7.23-7.34 (1H, m), 7.68-7.77 (2H, m), 7.80-7.91 (3H, m).

### Reference Example 23

### Synthesis of (1S,2R)-5,5-difluoro-2-({[4-(3-methyl-1H-pyrazol-1-yl)phenyl]carbonyl}amino)cyclohexanecarboxylic acid

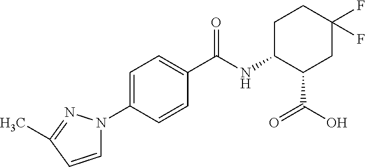

To a solution of ethyl (1S,2R)-5,5-difluoro-2({[4-(3-methyl-1H-pyrazol-1-yl)phenyl]carbonyl}amino)cyclohexanecarboxylate (154 g) in THF (1.5 L) was added dropwise an aqueous solution (1.0 L) of lithium hydroxide monohydrate (21.5 g) under ice-cooling, and the mixture was stirred at the same temperature for 3 hr. The reaction mixture was neutralized with 1N hydrochloric acid (500 mL), and extracted with ethyl acetate (1.5 L and 0.75 L). The combined extracts were washed with brine, dried over magnesium sulfate, and concentrated to give crude crystals. The obtained crude crystals were dissolved in tetrahydrofuran (720 mL) by heating (50-55° C.), and heptane (500 mL) was added dropwise at the same temperature. After stirring at the same temperature for 1 hr, heptane (220 mL) was further added dropwise to the reaction mixture and the mixture was stirred at 50° C. for 1 hr, at room temperature for 14 hr, and under ice-cooling for 1 hr. The precipitate was collected by filtration, washed with tetrahydrofuran/heptane mixed solvent (1:2), and dried to give the title compound (121 g) as pale-brown crystals.

¹H-NMR (DMSO-d₆) δ: 1.71-2.24 (5H, m), 2.29 (3H, s), 2.53-2.77 (1H, m), 2.90 (1H, dt, J=12.4, 3.9 Hz), 4.64-4.83 (1H, m), 6.38 (1H, d, J=2.3 Hz), 7.81-7.96 (4H, m), 8.25 (1H, d, J=9.0 Hz), 8.48 (1H, d, J=2.4 Hz), 12.49 (1H, brs).

### Reference Example 24

### Synthesis of {2-[(3aR*,4R*,9bR*)-1-{[(1S,2R)-5,5-difluoro-2-({[4-(3-methyl-1H-pyrazol-1-yl)phenyl]carbonyl}amino)cyclohexyl]carbonyl}-2,3,3a,4,5,9b-hexahydro-1H-pyrrolo[3,2-c]quinolin-4-yl]-1H-imidazol-1-yl}methyl 2,2-dimethylpropanoate

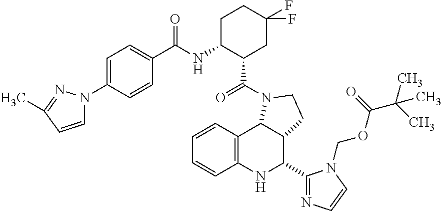

To a solution of {2-[(3aS*,4R*,9bR′)-2,3,3a,4,5,9b-hexahydro-1H-pyrrolo[3,2-c]quinolin-4-yl]-1H-imidazol-1-yl}methyl 2,2-dimethylpropanoate dihydrochloride (69.5 g), triethylamine (57.6 g), (1S,2R)-5,5-difluoro-2-({[4-(3-methyl-1H-pyrazol-1-yl)phenyl]carbonyl}amino)cyclohexanecarboxylic acid (59 g) in acetonitrile (1.7 L) were added N-[3-(dimethylamino)propyl]-N′-ethylcarbodiimide hydrochloride (33.4 g) and 1H-1,2,3-benzotriazol-1-ol (5.0 g) under ice-cooling, and the mixture was stirred at room temperature for 18 hr. To the reaction mixture was added 1H-1,2,3-benzotriazol-1-ol (5.0 g), and the mixture was stirred at room temperature for 22 hr. Water (0.5 L) and saturated aqueous sodium hydrogen carbonate solution (0.5 L) were added, and acetonitrile (1.0 L) was evaporated under reduced pressure. The organic layer was extracted with ethyl acetate (1.5 L), and the obtained organic layer was washed with brine (1 L), dried over magnesium sulfate, and concentrated under reduced pressure. The residue was purified by silica gel column chromatography (1.0 kg, elution solvent hexane:ethyl acetate=20:80) to give the title compound (109 g) as an amorphous solid.

¹H-NMR (CDCl₃) δ: 1.07-1.22 (9H, m), 1.88-2.02 (2H, m), 2.06-2.27 (2H, m), 2.32-2.88 (8H, m), 3.05-3.35 (1H, m), 3.44-3.79 (2H, m), 4.16-4.36 (1H, m), 4.52 (1H, d, J=15.1 Hz), 4.89-4.99 (1H, m), 5.70-6.10 (3H, m), 6.23-6.33 (1H, m), 6.46-6.65 (1H, m), 6.71-6.90 (1H, m), 6.98-7.26 (4H, m), 7.38-7.49 (1H, m), 7.66-8.08 (5H, m).

### Reference Example 25

### Synthesis of N-[(1R,2S)-4,4-difluoro-2-{[(3aR*,4R*,9bR*)-4-(1H-imidazol-2-yl)-2,3,3a,4,5,9b-hexahydro-1H-pyrrolo[3,2-c]quinolin-1-yl]carbonyl}cyclohexyl]-4-(3-methyl-1H-pyrazol-1-yl)benzamide

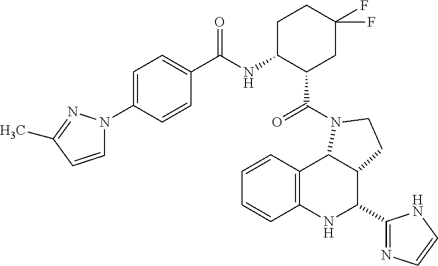

To a solution of {2-[(3aR*, 4R*,9bR*)-1-{[(1S,2R)-5,5-difluoro-2-({(4-(3-methyl-1H-pyrazol-1-yl)phenyl]carbonyl}amino)cyclohexyl]carbonyl}-2,3,3a,4,5,9b-hexahydro-1H-pyrrolo[3,2-c]quinolin-4-yl]-1H-imidazol-1-yl}methyl 2,2-dimethylpropanoate (109 g) in methanol (1.1 L) was added dropwise 28% aqueous ammonia (220 mL) at room temperature, and the mixture was stirred for 18 hr. Methanol was evaporated under reduced pressure, the residue was diluted with ethyl acetate (1 L) and water (1 L), and the insoluble material was filtered off. The filtrate was partitioned, and the aqueous layer was extracted again with ethyl acetate (1 L). The combined organic layer was washed with brine (1 L), dried over sodium sulfate, and concentrated under reduced pressure to give the title compound (86.4 g) as an amorphous solid.

¹H-NMR (CDCl₃) δ: 1.55-1.89 (2H, m), 1.92-2.26 (5H, m), 2.32-2.42 (3H, m), 2.44-2.76 (3H, m), 2.94-3.58 (3H, m), 4.22-5.79 (4H, m), 6.22-6.32 (1H, m), 6.33-6.47 (1H, m), 6.57-6.77 (1H, m), 6.86-7.16 (3H, m), 7.30-7.44 (1H, m), 7.55-8.01 (5H, m), 10.77-11.29 (1H, m).

### Reference Example 26

### Synthesis of {2-[(3aS,4S,9bS)-1-{[(1S,2R)-5,5-difluoro-2-({[4-(3-methyl-1H-pyrazol-1-yl)phenyl]carbonyl}amino)cyclohexyl]carbonyl}-2,3,3a,4,5,9b-hexahydro-1H-pyrrolo[3,2-c]quinolin-4-yl]-1H-imidazol-1-yl}methyl 2,2-dimethylpropanoate

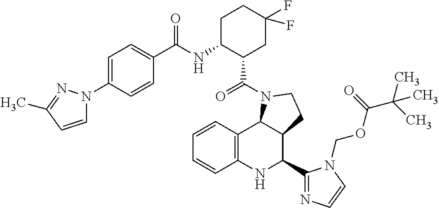

tert-Butyl (3aR*,4R*,9bR*)-4-(1-{[(2,2-dimethylpropanoyl)oxy]methyl}-1H-imidazol-2-yl)-2,3,3a,4,5,9b-hexahydro-1H-pyrrolo[3,2-c]quinoline-1-carboxylate was optically resolved using a chiral column, and the tert-butoxycarbonyl group was removed by a solution of 4N hydrogen chloride in ethyl acetate. The resulting {2-[(3aR,4S,9bS)-2,3,3a,4,5,9b-hexahydro-1H-pyrrolo[3,2-c]quinolin-4-yl]-1H-imidazol-1-yl}methyl 2,2-dimethylpropanoate dihydrochloride was reacted with (1S,2R)-5,5-difluoro-2-({[4-(3-methyl-1H-pyrazol-1-yl)phenyl]carbonyl}amino)cyclohexanecarboxylic acid in the same manner as in Reference Example 3 to synthesize the title compound.

LCMS, m/z 700(M+1)

### Example 1

### Synthesis of N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide

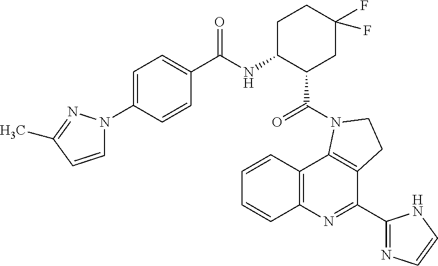

A mixture of N-[(1R,28)-4,4-difluoro-2-{[(3aR*,4R′,9bR*)-4-(1H-imidazol-2-yl)-2,3,3a,4,5,9b-hexahydro-1H-pyrrolo[3,2-c]quinolin-1-yl]carbonyl}cyclohexyl]-4-(3-methyl-1H-pyrazol-1-yl)benzamide (4.54 g), 2,3-dichloro-5,6-dicyano-p-benzoquinone (3.81 g) and tetrahydrofuran (35 mL) was stirred at 45-60° C. for 12 hr. To the reaction mixture was added 1N aqueous sodium hydroxide solution, and the mixture was extracted with ethyl acetate. The extracted organic layer was washed with 1N aqueous sodium hydroxide solution 3 times and with brine, dried over anhydrous sodium sulfate, and filtered by NH-silica gel column chromatography (elution solvent ethyl acetate). The filtrate was concentrated under reduced pressure, and the residue was purified by silica gel column chromatography (elution solvent ethyl acetate:hexane=10:90-100:0) and NH-silica gel column chromatography (elution solvent ethyl acetate). A mixture of the obtained product, activated carbon (650 mg) and ethyl acetate (100 mL) was stirred at room temperature for 1 hr, and filtered through celite. The filtrate was concentrated under reduced pressure, ethyl acetate, 2-propanol and hexane were added to the residue and the precipitate was collected by filtration to give the title compound (3.33 g) as a white amorphous solid.

LCMS, m/z 582(M+1)

¹H-NMR (CDCl₃) δ: 1.88-2.30 (3H, m), 2.30-2.52 (4H, m), 2.52-2.94 (2H, m), 3.49-3.68 (1H, m), 3.72-4.03 (2H, m), 4.39 (2H, t, J=8 Hz), 4.64 (1H, brs), 6.26 (1H, d, J=2.3 Hz), 7.15-7.36 (4H, m), 7.55-7.64 (1H, m), 7.64-7.75 (3H, m), 7.79-7.91 (3H, m), 7.99 (1H, d, J=8 Hz), 10.73 (1H, brs).

### Example 2

### Synthesis of N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrobromide

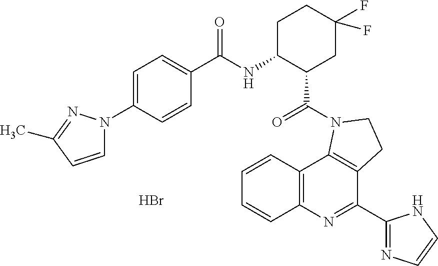

To a solution of N-[(1R,2S)-4,4-difluoro-2-([4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide (174 mg) in ethyl acetate (3 mL) was added an aqueous solution of hydrobromic acid (47%, 34.7 μL). Ethanol was added until the insoluble material was dissolved, and the mixture was concentrated under reduced pressure. The residue was crystallized from a mixture of ethanol and ethyl acetate, and washed with ethyl acetate to give the title compound (151 mg) as pale-yellow crystals.

¹H-NMR (DMSO-d₆) δ: 1.75-2.25 (4H, m), 2.30 (3H, s), 2.33-2.45 (1H, m), 2.67-3.01 (1H, m), 3.45-3.71 (2H, m), 3.71-3.88 (1H, m), 4.30-4.52 (1H, m), 4.86 (1H, t, J=9.1 Hz), 5.09 (1H, brs), 6.41 (1H, d, J=2.3 Hz), 6.99 (1H, t, J=7.7 Hz), 7.54 (1H, d, J=8.3 Hz), 7.65 (1H, t, J=7.7 Hz), 7.83 (2H, s), 7.92 (4H, s), 8.02 (1H, d, J=8.3 Hz), 8.52 (1H, d, J=2.3 Hz), 8.60 (1H, d, J=9.4 Hz).

Elemental analysis Calcd.: Br, 12.06. Found: Br, 12.14.

### Example 3

### Synthesis of N-[(1R,2S)-4,4-difluoro-2-{(4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrochloride

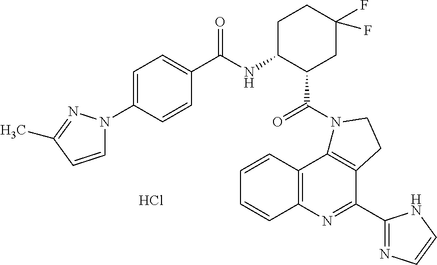

To a solution of N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide (222 mg) in ethanol (2 mL) was added a solution of 2N hydrogen chloride in ethanol (191 μL), and the mixture was stirred overnight and concentrated under reduced pressure to an about half amount. The mixture was stirred overnight to allow crystallization. Ethyl acetate was added and the mixture was further stirred for 1 hr. The resulting crystals were collected by filtration, washed with ethyl acetate, and dried under reduced pressure to give the title compound (120 mg) as pale-yellow crystals.

¹H-NMR (DMSO-d₆) δ: 1.78-2.24 (4H, m), 2.30 (3H, s), 2.35-2.48 (1H, m), 2.68-3.01 (1H, m), 3.48-3.71 (2H, m), 3.71-3.90 (1H, m), 4.39 (1H, q, J=9.8 Hz), 4.74-4.96 (1H, m), 5.08 (1H, brs), 6.41 (1H, d, J=2.3 Hz), 6.88-7.04 (1H, m), 7.53 (1H, d, J=7.9 Hz), 7.60-7.71 (1H, m), 7.78 (2H, s), 7.86-7.98 (4H, m), 8.03 (1H, d, J=8.3 Hz), 8.52 (1H, d, J=2.3 Hz), 8.61 (1H, d, J=9.8 Hz).

Elemental analysis Calcd.: Cl, 5.74. Found: Cl, 5.69.

### Example 4

### Synthesis of [2-(1-{[(1S,2R)-5,5-difluoro-2-({[4-(3-methyl-1H-pyrazol-1-yl)phenyl]carbonyl}amino)cyclohexyl]carbonyl)-2,3-dihydro-1H-pyrrolo[3,2-c]quinolin-4-yl)-1H-imidazol-1-yl]methyl 2,2-dimethylpropanoate

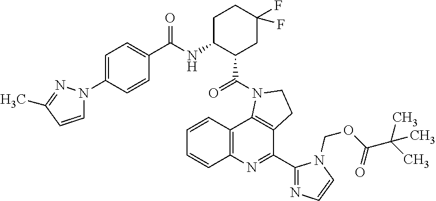

A mixture of {2-[(3aS,4S,9bS)-1-{[(1S,2R)-5,5-difluoro-2-({[4-(3-methyl-1H-pyrazol-1-yl)phenyl]carbonyl}amino)cyclohexyl]carbonyl}-2,3,3a,4,5,9b-hexahydro-1H-pyrrolo[3,2-c]quinolin-4-yl]-1H-imidazol-1-yl}methyl 2,2-dimethylpropanoate (350 mg), 2,3-dichloro-5,6-dicyano-p-benzoquinone (257.4 mg) and tetrahydrofuran (6 mL) was stirred at 45-60° C. for 12 hr. To the reaction mixture was added 0.1 N aqueous sodium hydroxide solution, and the mixture was extracted with ethyl acetate. The extracted organic layer was washed with 0.1 N aqueous sodium hydroxide solution, water and brine, dried over anhydrous sodium sulfate, and concentrated under reduced pressure. The residue was purified by silica gel column chromatography (elution solvent ethyl acetate:hexane=0:100-65:35) to give the title compound (317 mg) as a pale-brown amorphous solid.

LCMS, m/z 696(M+1)

¹H-NMR (CDCl₃) δ: 1.08 (9H, s), 1.87-2.29 (3H, m), 2.29-2.51 (4H, m), 2.51-2.88 (2H, m), 3.57 (1H, dt, J=7.1, 4.8 Hz), 3.71-3.90 (2H, m), 4.35 (2H, t, J=8.1 Hz), 4.63 (1H, brs), 6.26 (1H, d, J=2.3 Hz), 6.61-6.78 (2H, m), 7.20 (1H, s), 7.25 (1H, s), 7.31 (2H, dt, J=7.2, 3.6 Hz), 7.56-7.64 (1H, m), 7.64-7.73 (3H, m), 7.80-7.92 (3H, m), 8.00 (1H, d, J=7.9 Hz).

### Example 5

### Synthesis of N-[(1R,2S)-4,4-difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide

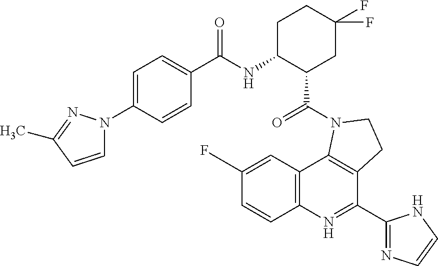

A mixture of N-[(1R,2S)-4,4-difluoro-2-{[(3aR′,4R*,9bR*)-8-fluoro-4-(1H-imidazol-2-yl)-2,3,3a,4,5,9b-hexahydro-1H-pyrrolo[3,2-c]quinolin-1-yl]carbonyl}cyclohexyl]-4-(3-methyl-1H-pyrazol-1-yl)benzamide (200 mg), manganese dioxide (2.02 g) and toluene (100 mL) was stirred at 80° C. for 12 hr. The reaction mixture was filtered through celite, and the filtrate was concentrated. The residue was purified by silica gel column chromatography (elution solvent ethyl acetate:hexane=30:70-100:0) to give the title compound (50.0 mg) as a white amorphous solid.

LCMS, m/z 600(M+1)

¹H-NMR (CDCl₃) δ: 1.94-2.50 (7H, m), 2.51-2.81 (2H, m), 3.48-3.62 (1H, m), 3.74-3.93 (2H, m), 4.30-4.45 (2H, m), 4.66 (1H, s), 6.26 (1H, d, J=2.3 Hz), 7.17-7.28 (2H, m), 7.33-7.44 (3H, m), 7.68 (2H, d, J=9.1 Hz), 7.74-7.91 (3H, m), 7.98 (1H, dd, J=9.3, 5.5 Hz).

### Example 6

### Synthesis of N-[(1R,2S)-4,4-difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrochloride

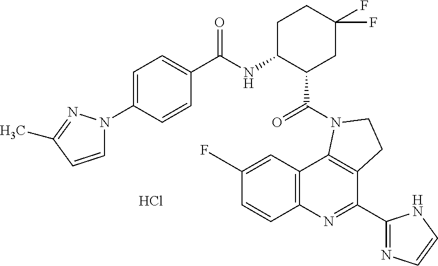

To a solution of N-[(1R,25)-4,4-difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide (5 mg) in methanol (0.1 mL) was added a solution of hydrogen chloride in methanol (2N, 10 μL) and the solvent was evaporated under reduced pressure. To the residue was added ethyl acetate and the crystals were collected by filtration and dried under reduced pressure to give the title compound (6.4 mg) as a pale-yellow amorphous solid.

LCMS, m/z 600(M+1)

### Example 7

### Synthesis of N-[(1R,2S)-4,4-difluoro-2-{(4-thiophen-3-yl-2,3-dihydro-1H-pyrrolo[3,2-c]quinolin-1-yl)carbonyl]cyclohexyl}-4-(3-methyl-1H-pyrazol-1-yl)benzamide

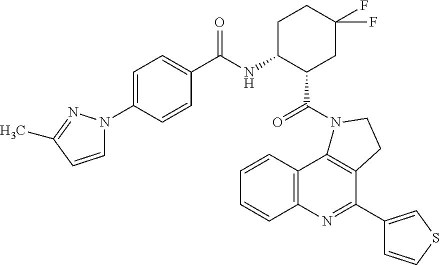

Using N-[(1R,2S)-4,4-difluoro-2-{[(3aR*,4R*,9bR*)-4-thiophen-3-yl-2,3,3a,4,5,9b-hexahydro-1H-pyrrolo[3,2-c]quinolin-1-yl]carbonyl}cyclohexyl]-4-(3-methyl-1H-pyrazol-1-yl)benzamide (155 mg) and in the same manner as in Example 5, the title compound (45 mg) was obtained as a white amorphous solid.

LCMS, m/z 598(M+1)

¹H-NMR (CDCl₃) δ: 1.85-2.30 (3H, m), 2.31-2.50 (4H, m), 2.52-2.85 (2H, m), 3.35-3.65 (3H, m), 4.33-4.44 (2H, m), 4.61 (1H, s), 6.21-6.35 (1H, m), 7.28-7.36 (1H, m), 7.39-7.48 (1H, m), 7.58-7.91 (10H, m), 8.05-8.15 (1H, m).

### Example 8

### Synthesis of N-1(1R,2S)-4,4-difluoro-2-[(4-thiophen-3-yl-2,3-dihydro-1H-pyrrolo[3,2-c]quinolin-1-yl)carbonyl]cyclohexyl}-4-(3-methyl-1H-pyrazol-1-yl)benzamide hydrochloride

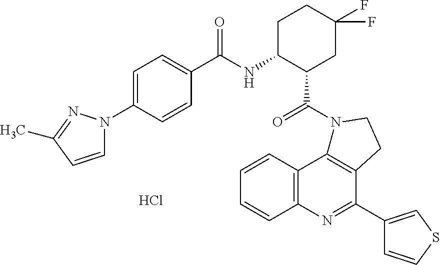

To a solution of N-{(1R,2S)-4,4-difluoro-2-[(4-thiophen-3-yl-2,3-dihydro-1H-pyrrolo[3,2-c]quinolin-1-yl)carbonyl]cyclohexyl}-4-(3-methyl-1H-pyrazol-1-yl)benzamide (5 mg) in methanol (0.1 mL) was added a solution of hydrogen chloride in methanol (2N, 10 μL), and the solvent was evaporated under reduced pressure. To the residue was added ethyl acetate and the crystals were collected by filtration and dried under reduced pressure to give the title compound (6.2 mg) as a pale-yellow amorphous solid.

LCMS, m/z 598(M+1)

### Example 9

### Synthesis of N-{(1R,2S)-4,4-difluoro-2-[(8-fluoro-4-phenyl-2,3-dihydro-1H-pyrrolo[3,2-c]quinolin-1-yl)carbonyl]cyclohexyl}-4-(3-methyl-1H-pyrazol-1-yl)benzamide

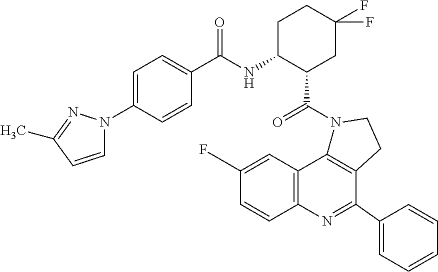

Using N-[(1R,2S)-4,4-difluoro-2-{[(3aR*, 4R*,9bR*)-8-fluoro-4-phenyl-2,3,3a,4,5,9b-hexahydro-1H-pyrrolo[3,2-c]quinolin-1-yl]carbonyl}cyclohexyl]-4-(3-methyl-1H-pyrazol-1-yl)benzamide (205 mg) and in the same manner as in Example 5, the title compound (60 mg) was obtained as a white amorphous solid.

LCMS, m/z 610(M+1)

¹H-NMR (CDCl₃) δ: 1.91-2.32 (2H, m), 2.32-2.50 (4H, m), 2.68 (2H, d, J=32.4 Hz), 3.35 (1H, s), 3.53 (2H, d, J=8.9 Hz), 4.24-4.41 (2H, m), 4.65 (1H, s), 6.26 (1H, d, J=2.4 Hz), 7.23 (1H, d), 7.35-7.55 (5H, m), 7.66-7.72 (2H, m), 7.78 (2H, dd, J=7.9, 1.5 Hz), 7.81-7.89 (3H, m), 8.15 (1H, d, J=9.0 Hz).

### Example 10

### Synthesis of N-{(1R,2S)-4,4-difluoro-2-[(8-fluoro-4-phenyl-2,3-dihydro-1H-pyrrolo[3,2-c]quinolin-1-yl)carbonyl]cyclohexyl}-4-(3-methyl-1H-pyrazol-1-yl)benzamide hydrochloride

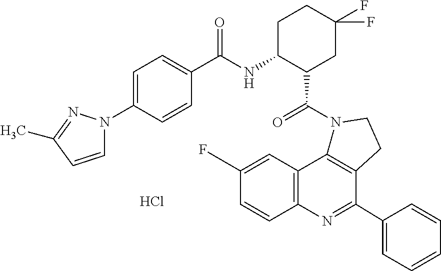

To a solution of N-{(1R,2S)-4,4-difluoro-2-[(8-fluoro-4-phenyl-2,3-dihydro-1H-pyrrolo[3,2-c]quinolin-1-yl)carbonyl]cyclohexyl}-4-(3-methyl-1H-pyrazol-1-yl)benzamide (5 mg) in methanol (0.1 mL) was added a solution of hydrogen chloride in methanol (2N, 10 μL), and the solvent was evaporated under reduced pressure. To the residue was added ethyl acetate and the crystals were collected by filtration, and dried under reduced pressure to give the title compound (6.0 mg) as a pale-yellow amorphous solid.

LCMS, m/z 610(M+1)

### Example 11

### Synthesis of N-[(1R,2S)-4,4-difluoro-2-{(4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide

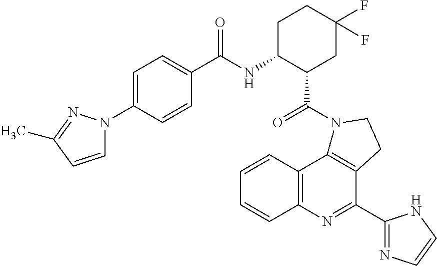

To a solution of 2,3,5,6-tetrachloro-1,4-benzoquinone (78 g) in acetonitrile (1.34 L) was added at 55° C. N-[(1R,2S)-4,4-difluoro-2-{[(3aR*,4R*,9bR*)-4-(1H-imidazol-2-yl)-2,3,3a,4,5,9b-hexahydro-1H-pyrrolo[3,2-c]quinolin-1-yl]carbonyl}cyclohexyl]-4-(3-methyl-1H-pyrazol-1-yl)benzamide (84 g) obtained in Reference Example 25, and the mixture was stirred for 2.5 hr. The reaction mixture was cooled to room temperature, and a mixture of water (0.5 L) and saturated aqueous sodium hydrogen carbonate solution (0.5 L) was added dropwise. The insoluble material was filtered off and washed with acetonitrile. The filtrate and washing were combined, and acetonitrile in the mixture was evaporated under reduced pressure. The aqueous layer was diluted with water (840 mL), and the organic layer was extracted with ethyl acetate (1.5 L and 0.75 L). The combined organic layers were washed with brine and dried over sodium sulfate, and ethyl acetate (about 1.75 L) was evaporated under reduced pressure. The residue was purified by NH-silica gel column chromatography (1.3 kg, elution solvent ethyl acetate) to give the title compound (71 g) as an amorphous solid.

¹H-NMR (CDCl₃) δ: 1.90-2.03 (1H, m), 2.06-2.26 (2H, m), 2.27-2.47 (1H, m), 2.35 (3H, s), 2.50-2.79 (2H, m), 3.48-3.61 (1H, m), 3.69-3.90 (2H, m), 4.24-4.41 (2H, m), 4.66 (1H, brs), 6.24 (1H, d, J=2.3 Hz), 7.12 (1H, d, J=1.1 Hz), 7.20-7.29 (2H, m), 7.34 (1H, d, J=7.5 Hz), 7.54 (1H, ddd, J=8.4, 6.9, 1.4 Hz), 7.61-7.71 (3H, m), 7.77-7.88 (3H, m), 7.91 (1H, d, J=7.9 Hz), 11.22 (1H, brs).

### Example 12

### Synthesis of N-[(1R,2S)-4,4-difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide hydrochloride

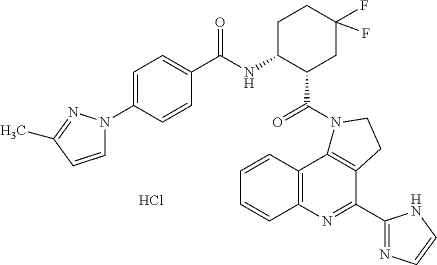

N-[(1R,2S)-4,4-Difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide (130 g) was dissolved in ethanol (260 mL) and isopropyl acetate (650 mL), and a solution of 4N hydrogen chloride in ethyl acetate (62 mL), which had been diluted with isopropyl acetate (650 mL), was added dropwise under ice-cooling. The reaction mixture was stirred under ice-cooling for 15 min, isopropyl acetate (650 mL) was added dropwise thereto, and the mixture was stirred at room temperature for 30 min, at 60° C. for 2.5 hr, and at room temperature for 1 hr. The precipitate was collected by filtration, and washed with ethanol/isopropyl acetate mixed solvent (1:8, 1.3 L) to give crude crystals (117 g). To a mixture of the thus-obtained crude crystals (169 g) and 5% water/ethanol mixed solvent (1.69 L) was added water (1.5 mL), and the mixture was heated to 60° C. to dissolve the crude crystals. The insoluble material was filtered off, and washed with 5% water/ethanol mixed solution (169 mL). The filtrate and washing were combined, and the mixture was dissolved by heating at 60° C. tert-Butyl methyl ether (1.5 L) was added dropwise at the same temperature and the mixture was stirred for 2 hr. To this mixture was further added dropwise tert-butyl methyl ether (1.5 L), and the mixture was stirred for 30 min, and at room temperature for 1 hr. The precipitate was collected by filtration, washed with ethanol/tert-butyl methyl ether mixed solvent (1:2, 1.0 L), and dried to give the title compound (157 g) as pale-yellow crystals.

LCMS, m/z. 582 (M+1)

¹H-NMR (DMSO-d₆) δ: 1.80-2.26 (4H, m), 2.30 (3H, s), 2.33-2.48 (1H, m), 2.68-3.02 (1H, m), 3.48-3.73 (2H, m), 3.75-3.95 (1H, m), 4.38 (1H, q, J=9.8 Hz), 4.76-4.96 (1H, m), 5.10 (1H, brs), 6.41 (1H, d, J=2.3 Hz), 6.91-7.06 (1H, m), 7.54 (1H, d, J=7.9 Hz), 7.66 (1H, ddd, J=8.5, 7.0, 1.3 Hz), 7.82 (2H, s), 7.88-7.98 (4H, m), 8.04 (1H, d, J=8.3 Hz), 8.53 (1H, d, J=2.4 Hz), 8.63 (1H, d, J=9.8 Hz).

Water content measurement (coulometric titration method): 0.68% (humidity 42.3%)

Elemental analysis: C₃₂H₃₀ClF₂N₇O₂.0.23H₂O

Calcd. C, 61.77; H, 4.93; N, 15.76; Cl, 5.70

Found C, 61.70; H, 4.95; N, 15.82; Cl, 5.67

Melting point: 232° C.

[α]_{D}²⁵−230.6° (c 0.27, MeOH)

powder X-ray: The powder X-ray crystal diffraction pattern of the present crystals (Form A) measured using Cu-Kα ray (tube voltage: 40 KV; tube electric current: 50 mA) as a radiation source and RINT Ultima+2100 powder X-ray diffraction apparatus (manufactured by Rigaku Corporation) is shown in Table 1.

30

TABLE 1
Data of powder X-ray crystal diffraction (main peaks)

| Diffraction | Spacing: |
|---|---|
| angle: | d value |
| 2θ (°) | (angstrom) |
| 4.12 | 21.4288 |
| 8.48 | 10.4184 |
| 8.9 | 9.9277 |
| 10.98 | 8.0513 |
| 12.64 | 6.9974 |
| 13.9 | 6.3658 |
| 15.64 | 5.6613 |
| 15.98 | 5.5416 |
| 16.6 | 5.336 |
| 17.86 | 4.9623 |
| 18.34 | 4.8335 |
| 19.34 | 4.5857 |
| 19.68 | 4.5073 |
| 20.76 | 4.2752 |
| 21.28 | 4.1719 |
| 22.48 | 3.9518 |
| 23.52 | 3.7794 |
| 23.98 | 3.7079 |
| 24.7 | 3.6014 |
| 25.2 | 3.5311 |
| 25.46 | 3.4956 |
| 26.72 | 3.3336 |
| 27.06 | 3.2924 |
| 28.02 | 3.1818 |
| 28.78 | 3.0995 |
| 30.8 | 2.9006 |
| 32.32 | 2.7676 |

### Formulation Example 1

### (1) Compound of Example 110.0 g

### (2) Lactose 70.0 g

### (3) Cornstarch 50.0 g

(4) Soluble starch 7.0 g
(5) Magnesium stearate 3.0 g

The compound of Example 1 (10.0 g) and magnesium stearate (3.0 g) are granulated with an aqueous solution of soluble starch (70 mL, 7.0 g as soluble starch), dried and mixed with lactose (70.0 g) and cornstarch (50.0 g) (lactose, cornstarch, soluble starch and magnesium stearate are all products on the Japanese Pharmacopoeia 14th ed. or Japanese Pharmaceutical Excipients). The mixture is compressed to give tablets.

### Experimental Example 1

### Radioligand Receptor Binding Inhibitory Activity Using Membrane Fraction of hNK2 Receptor-Expressing CHO Cell

hNK2 receptor-expressing CHO cells (produced by EUROSCREEN) were cultured in a flask placed HAM-F12 medium containing 400 μg/mL Geneticin, 100 U/mL penicillin, 100 μg/mL streptomycin and 10% inactivated serum. The medium was removed and adhered cells were washed with PBS, and PBS containing 5 mM EDTA was added to detach the cells from the flask. The cells were recovered by centrifugation, suspended in suspending buffer A (15 mM Tris-HCl (pH 7.5), 12.5 mM MgCl₂, 0.3 mM EDTA, 1 mM EGTA (Ethylene Glycol Bis(beta-aminoethylether)-N,N,N,N-tetraacetic Acid)), disrupted by POLYTRON homogenizer (manufactured by KINEMATICA) and centrifuged at 850×g for 10 min at 4° C. The supernatant was recovered and ultracentrifuged at 142000×g for 60 min at 4° C. The precipitated fraction was suspended in suspending buffer B (7.5 mM Tris-HCl (pH 7.5), 12.5 mM MgCl₂, 0.3 mM EDTA, 1 mM EGTA, 250 mM sucrose), and cryopreserved (at −80° C.) as a membrane fraction preparation of hNK2 receptor-expressing CHO cell.

### [Receptor Binding Assay]

The radioligand receptor binding assay was performed in a 96 well microassay plate format with a final volume of 200 μL.

An assay buffer (50 μL, 50 mM Tris-HCl (pH 7.4), 0.02% bovine serum albumin, 2 μg/mL chymostatin, 40 μg/mL bacitracin, 40 μg/mL APMSF, 3 mM MnCl₂) was added to a 96 well microassay plate. Thereto was added 50 μL of the above-mentioned cryopreserved membrane fraction preparation suspended in the assay buffer (20 μg/mL). An assay buffer (50 μL) containing 2% dimethyl sulfoxide was added to examine the total binding, 4 μM unlabeled NKA (produced by Peptide Institute, Inc.) solution (50 μL) diluted with an assay buffer containing 2% dimethyl sulfoxide was added to examine non-specific binding, and a test compound diluted with an assay buffer (50 μL, containing 2% dimethyl sulfoxide) was added. Furthermore, 400 μM [¹²⁵I]-NKA (produced by NEN) solution (50 μL) was added to each well.

After reaction at room temperature for 30 min, using a cell harvester (manufactured by PerkinElmer), the reaction was stopped by rapid filtration on a unifilter plate (GF/C) (manufactured by PerkinElmer), which had been immersed in 0.3% aqueous polyethyleneimine solution for one day, and the cells were washed 10 times with 50 mM Tris-HCl (pH 7.4) buffer (250 μL) containing 0.02% bovine serum albumin. The GF/C filter plate was dried and 20 μL of MicroScinti-0 (manufactured by PerkinElmer) was added to each well and the radioactivity was measured by TopCount (manufactured by PerkinElmer). The results are shown in Table 2.

Specific binding is shown by the value obtained by subtracting non-specific binding from the total binding. The binding inhibitory activity of the test compound is shown by the ratio of the value obtained by subtracting the measured value associated with the addition of the test compound from the total binding, to the value of the specific binding.

TABLE 2
hNK2 receptor binding inhibitory activity (inhibitory rate (%))

| Test compound | 10⁻⁵ M | 10⁻⁶ M | 10⁻⁷ M | 10⁻⁸ M |
|---|---|---|---|---|
| Example 1 | NT | 101.1 | 100.5 | 99.7 |
| Example 2 | NT | 100.7 | 99.7 | 99.6 |
| Example 3 | NT | 101.7 | 101.4 | 100.3 |
| Example 4 | NT | 80.1 | 31.6 | −7.5 |
| Example 5 | NT | 100.8 | 99.8 | 96.6 |
| Example 6 | NT | 99.4 | 99.4 | 96.1 |
| Example 7 | 101.7 | 100.3 | 100.1 | 98.8 |
| Example 8 | NT | 100.7 | 100.2 | 98.5 |
| Example 9 | 99.1 | 100.6 | 99.9 | 93.8 |
| Example 10 | NT | 99.9 | 99.5 | 92.8 |
| Example 12 | NT | 100.9 | 101.4 | 99.8 |

NT: not tested

### Experimental Example 2

### Evaluation of Antagonistic Activity Determined by Intracellular Calcium Concentration Using Test Compound, Neurokinin A and hNK2 Receptor-Expressing CHO Cell

hNK2 receptor-expressing CHO cells were seeded on a 384 well plate at 7.5×10³ cells/well and cultured for 24 hr. Then the medium was removed, 1× recording medium (30 μL, Calcium kit II-Fluo 4: DOJINDO LABORATORIES, Japan) containing prepared 2.5 μg/mL Fluo 4, 2.5 mM probenecid and 20 mM HEPES was added, and the cells were cultured in CO2 incubator (37° C., 5% CO2) for 1 hr. A test compound diluted with 1× recording medium containing 0.05% bovine serum albumin and 20 mM HEPES (10 μL, 0.6% dimethyl sulfoxide) was added. Thereafter, 3 nM neurokinin A solution (20 μL) was added, and changes in the intracellular calcium concentration were measured by FLIPR (Molecular Devices Corporation, Japan). The inhibitory rate of the test compound was calculated with a reaction using DMSO alone and free of the compound as 100% inhibition and a reaction using 1 nM NKA as 0% inhibition. The results are shown in Table 3.

TABLE 3
Antagonistic activity (inhibitory rate (%))

| Test compound | 10⁻⁵ M | 10⁻⁶ M | 10⁻⁷ M | 10⁻⁸ M |
|---|---|---|---|---|
| Example 1 | 88.3 | 102.0 | 102.5 | 100.1 |
| Example 2 | 88.8 | 102.2 | 102.8 | 100.4 |
| Example 3 | 86.8 | 102.5 | 102.9 | 100.5 |
| Example 4 | 91.0 | 98.7 | 39.5 | −6.8 |
| Example 5 | 87.1 | 100.2 | 102.1 | 100.2 |
| Example 7 | 83.7 | 99.3 | 100.5 | 98.6 |
| Example 9 | 84.1 | 96.7 | 100.5 | 81.9 |

### Experimental Example 3

### Radioligand Receptor Binding Inhibitory Activity Using Membrane Fraction of hNK3 Receptor-Expressing CHO Cell

### [Preparation of Membrane Fraction]

hNK3 receptor-expressing CHO cells were cultured in a HAM-F12 medium containing 500 μg/mL Geneticin, 100 U/mL penicillin, 100 μg/mL streptomycin and 10% inactivated serum. The medium was removed before reaching confluence, and the cells were washed with PBS. PBS containing 0.5 mM EDTA was added to detach the cells from the flask and a cell suspension was recovered. The cells were recovered by low speed centrifugation. The cells were suspended in suspending buffer A (50 mM Tris-HCl (pH 7.4), 120 mM NaCl, 5 mM KCl, bacitracin (40 μg/mL), chymostatin (2 μg/mL), PMSF (0.5 mM), 1 mM EDTA), disrupted by a POLYTRON homogenizer (manufactured by KINEMATICA) and centrifuged at 2000×g for 10 min at 4° C. The supernatant was recovered, and ultracentrifuged at 40000×g for 60 min at 4° C. The precipitated fraction was suspended in suspending buffer B (50 mM Tris-HCl (pH 7.4), 3 mM MnCl₂, 0.02% BSA, bacitracin (40 μg/mL), chymostatin (2 μg/mL), PMSF (0.5 mM)). The protein concentration of the suspension was measured using Bio-Rad Protein Assay kit and bovine serum albumin (protein preparation), and cryopreserved (at −80° C.) as a membrane fraction preparation of hNK3 receptor-expressing CHO cell until use for a binding assay.

### [Receptor Binding Assay]

The radioligand receptor binding assay was performed in a 96 well microassay plate format with a final volume of 200 μL. To the 96 well microassay plate were added 50 μL of assay buffer (50 mM Tris-HCl (pH 7.4), 0.02% bovine serum albumin, 2 μg/mL chymostatin, 40 μg/mL bacitracin, 40 μg/mL APMSF, 3 mM MnCl₂), the aforementioned cryopreserved membrane fraction preparation suspended in assay buffer (50 μL (300 μg/mL)) and a test compound diluted with 50 μL of assay buffer (containing 2% dimethyl sulfoxide). The reaction was started by the addition of 50 μL of 400 pM [¹²⁵I]-NKB NKB (PerkinElmer Life Sciences) solution. For the measurement of the total binding, assay buffer (50 μL) containing 2% dimethyl sulfoxide was added instead of a diluted test compound solution, and for the measurement of non-specific binding, 50 μL of 4 μM unlabeled NKB (manufactured by Peptide Institute, Inc.) solution (diluted with assay buffer containing 2% dimethyl sulfoxide) was added.

After reaction at room temperature for 30 min, using a cell harvester (manufactured by PerkinElmer), the reaction was stopped by rapid filtration on a unifilter plate (GF/C) (manufactured by PerkinElmer), which had been immersed in 0.3% polyethyleneimine for one day, and the membrane fraction was washed 10 times with 50 mM Tris-HCl (pH 7.4) buffer containing 0.02% bovine serum albumin (250 μL). The GF/C filter plate was dried and 20 μL of MicroScinti-O (manufactured by PerkinElmer) was added to each well and the radioactivity was measured by TopCount (manufactured by PerkinElmer). The results are shown in Table 4.

Specific binding is shown by the value obtained by subtracting non-specific binding from the total binding. The binding inhibitory activity of the test compound is shown by the ratio of the value obtained by subtracting the measured value associated with the addition of the test compound from the total binding, to the value of the specific binding.

TABLE 4
hNK3 receptor binding inhibitory activity (inhibitory rate (%))

| Test compound | 10⁻⁵ M | 10⁻⁶ M |
|---|---|---|
| Example 1 | 28.4 | 9.0 |
| Example 2 | 32.7 | 4.1 |
| Example 3 | 30.4 | 9.4 |
| Example 4 | −7.9 | −6.1 |
| Example 5 | 15.2 | −8.1 |
| Example 7 | 35.6 | 5.9 |
| Example 9 | 53.1 | 27.7 |

### INDUSTRIAL APPLICABILITY

The present invention enables provision of a dihydro pyrroloquinoline derivative, which is compound (I) useful as an NK receptor, particularly NK2 receptor antagonist, and can provide a pharmaceutical product showing a high treatment effect as an NK receptor, particularly NK2 receptor antagonist.

This application is based on patent application Nos. 2009-255574 and 2010-94144 filed in Japan, the contents of which are all hereby incorporated by reference.

The disclosure of all references cited herein are incorporated herein in their entireties by reference.

The invention has been described in detail with reference to certain embodiments. However, it will be appreciated that those skilled in the art, upon consideration of this disclosure, may make modifications and improvements within the spirit and scope of the invention. Any and all such modifications, improvements and/or equivalents are intended to be encompassed by the following claims.

1. A compound represented by the formula (I) 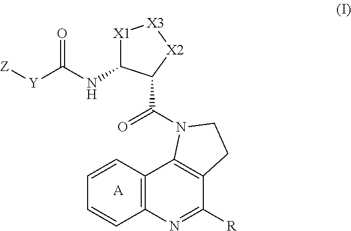
wherein
A is a benzene ring optionally having substituent(s),
R is a hydrogen atom, a hydrocarbon group optionally having substituent(s) or a heterocyclic group optionally having substituent(s),
X1 and X2 are each a bond or a divalent C₁₋₅ chain hydrocarbon group optionally having substituent(s),
X3 is a methylene group having substituent(s),
Y is a bond or an imino group (—NH—) optionally having a substituent, and
Z is a hydrocarbon group optionally having substituent(s) or a heterocyclic group optionally having substituent(s), or a salt thereof.

2. The compound according to claim 1, wherein X3 is a methylene group having substituent(s) selected from (1) a fluorine atom and (2) a C₁₋₃ alkyl group substituted by fluorine atom(s), or a salt thereof.

3. The compound according to claim 1, wherein X1 is ethylene (—CH₂CH₂—) and X2 is methylene (—CH₂—), or a salt thereof.

4. The compound according to claim 2, wherein X3 is a methylene group having fluorine atom(s), or a salt thereof.

5. The compound according to claim 1, wherein R is an aromatic hydrocarbon group optionally having substituent(s) or an aromatic heterocyclic group optionally having substituent(s), or a salt thereof.

6. The compound according to claim 1, wherein A is a benzene ring optionally substituted by fluorine atom(s), or a salt thereof.

7. The compound according to claim 1, wherein Y is a bond or an imino group (—NH—), or a salt thereof.

8. The compound according to claim 1, wherein Z is an aromatic hydrocarbon group optionally having substituent(s) or an aromatic heterocyclic group optionally having substituent(s), or a salt thereof.

9. N-[(1R,2S)-4,4-Difluoro-2-{[4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide or a salt thereof.

10. N-[(1R,2S)-4,4-Difluoro-2-{[8-fluoro-4-(1H-imidazol-2-yl)-2,3-dihydro-1H-pyrrolo[3,2-c]quinolin-1-yl]carbonyl}cyclohexyl]-4-(3-methyl-1H-pyrazol-1-yl)benzamide or a salt thereof.

11. N-{(1R,2S)-4,4-Difluoro-2-[(8-fluoro-4-phenyl-2,3-dihydro-1H-pyrrolo[3,2-c]quinolin-1-yl)carbonyl}cyclohexyl]-4-(3-methyl-1H-pyrazol-1-yl)benzamide or a salt thereof.

12. A prodrug of the compound according to claim 1 or a salt thereof.

13. A neurokinin (NK) receptor antagonist comprising the compound according to claim 1 or a salt thereof, or a prodrug thereof.

14. A neurokinin 2 (NK2) receptor antagonist comprising the compound according to claim 1 or a salt thereof, or a prodrug thereof.

15. A medicament comprising the compound according to claim 1 or a salt thereof, or a prodrug thereof.

16. The medicament according to claim 15, which is an agent for the prophylaxis or treatment of a gastrointestinal disease or a central nervous system disease.

17. The medicament according to claim 16, wherein the gastrointestinal disease is a functional gastrointestinal disease.

18. The medicament according to claim 17, wherein the functional gastrointestinal disease is irritable bowel syndrome or functional dyspepsia.

19. A method for preventing or treating a gastrointestinal disease or a central nervous system disease, comprising administering an effective amount of the compound according to claim 1 or a salt thereof, or a prodrug thereof to a mammal.

20. The method according to claim 19, wherein the gastrointestinal disease is a functional gastrointestinal disease.

21. The method according to claim 20, wherein the functional gastrointestinal disease is irritable bowel syndrome or functional dyspepsia.

22. Use of the compound according to claim 1 or a salt thereof, or a prodrug thereof for the production of an agent for the prophylaxis or treatment of a gastrointestinal disease or a central nervous system disease.

23. The use according to claim 22, wherein the gastrointestinal disease is a functional gastrointestinal disease.

24. The use according to claim 23, wherein the functional gastrointestinal disease is irritable bowel syndrome or functional dyspepsia.

25. A method for antagonizing an NK2 receptor, comprising administering an effective amount of the compound according to claim 1 or a salt thereof, or a prodrug thereof to a mammal.

26. Use of the compound according to claim 1 or a salt thereof, or a prodrug thereof for the production of an NK2 receptor antagonist.

27. The compound according to claim 1 or a salt thereof, or a prodrug thereof for use in the prophylaxis or treatment of a gastrointestinal disease or a central nervous system disease.
